(19) United States
(12) Patent Application Publication
(10) Pub. No.: US 2022/0106596 A1
(43) Pub. Date: Apr. 7, 2022

(54) A METHOD OF GENE EDITING

(71) Applicant: University of Washington, Seattle, WA (US)

(72) Inventors: David W. RUSSELL, Seattle, WA (US); Francoise J. HAESELEER, Seattle, WA (US); Dhwanil DALWADI, Seattle, WA (US)

(21) Appl. No.: 17/423,236

(22) PCT Filed: Jan. 29, 2020
(86) PCT No.: PCT/US2020/015550

§ 371 Date: Jul. 15, 2021

Related U.S. Application Data
(60) Provisional application No. 62/798,357, filed on Jan. 29, 2019.

(51) Int. Cl. C12N 15/113; C12N 15/86; C12N 9/90; A61K 48/00; A61K 35/12

(52) U.S. Cl. CPC C12N 15/113; C12N 15/86; C12N 9/90; C12N 2750/14143; A61K 35/12; C12N 2310/14; A61K 48/0066

(57) ABSTRACT

Disclosed herein are methods of editing a gene in a cell that involve contacting the cell with a replication fork modulator, as well as edited cells and their methods of use.

### CROSS REFERENCE

This application claims priority to U.S. Provisional Patent Application Ser. No. 62/798,357 filed Jan. 29, 2019, incorporated by reference herein in its entirety.

### FEDERAL FUNDING STATEMENT

This invention was made with government support wider Grant No. R01 DK055759, awarded by the National institutes of Health (NIH). The government has certain rights in the invention

### FIELD OF THE INVENTION

The invention relates to methods of gene editing, production of edited cells and their method of use.

### BACKGROUND OF THE INVENTION

The replication fork (RF) is a multiprotein complex with helicase and DNA synthesis activities. The replication fork has two branching “prongs”, each one made up of a single strand of DNA. These two strands serve as the template for leading and lagging strand DNA synthesis. The replicative helicase unwinds the parental duplex DNA exposing two ssDNA templates. DNA polymerases perform DNA synthesis. Because of the antiparallel nature of duplex DNA, DNA replication occurs in opposite directions between the two new strands at the replication fork. DNA synthesis is mediated by DNA polymerases. The strand that is synthesized in the same direction as that of the moving replication fork, the leading strand, is replicated continuously, whereas the strand synthesized in the opposite direction, the lagging strand, is replicated discontinuously. The coordinated and regulated activities of the numerous proteins associated with the replication fork mediate DNA replication. (Reviewed in Waga and Stillman, 1998, Annu. Rev. Biochem. 67:721-51 and Burgers and Kunkel, 2017 Annu. Rev. Biochem. 86: 417-438)

### SUMMARY OF THE INVENTION

The invention provides for a method of editing a gene in a cell comprising modulating replication fork function in the cell, and editing the gene in the cell.

In one embodiment, the method further comprises contacting the cell with a replication fork modulator.

In another embodiment, the method further comprises contacting the cell with a gene editing vector.

In another embodiment, the gene editing vector is an adeno-associated virus (AAV) vector.

In another embodiment, the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

In another embodiment, the replication fork modulator is emetine. In certain embodiments, emetine is used at a concentration of between 1 nM to 100,000 nM, for example, 10 nM to 10,000 nM, 100 nM-10,000 nM, 200 nM-10,000 nM, 300 nM-10,000 nM, 400 nM-10,000 nM, 500 nM to 10,000 nM, or between 100 nM and 10,000 nM, or between 100 nM and 1000 nM, for example, 100 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM or 950 nM, or between 1000 nM and 10,000 nM, for example, 1000 nM, 2000 nM, 3000 nM, 4000 nM, 5000 nM, 6000 nM, 7000 nM, 8000 nM, 9000 nM and 10,000 nM.

In another embodiment, the replication fork modulator is an siRNA.

In another embodiment, the replication fork modulator is an shRNA

In another embodiment, the replication fork function is DNA synthesis.

In another embodiment, the replication fork modulator is a leading strand synthesis inhibitor.

In another embodiment, the replication fork modulator is a lagging strand synthesis inhibitor.

In another embodiment, the method further comprises modulating the function or level of expression of a replication fork protein.

In another embodiment, the replication fork protein is selected from the group consisting of: DNA polymerase α, DNA primase, RNA primase, DNA polymerase ε, DNA polymerase δ, fork protection complex (FPC) components Timeless, Tipin, Claspin and And1, Cdc45, MCM 2-7 (mini-chromosome maintenance) helicase 2-7 hexamer proteins (Mcm2, Mcm3, Mcm4, Mcm5, Mcm6 and Mcm7), go-ichi-ni-san (GINS) complex proteins (Sld5, Psf1, Psf2 and Psf3), replication protein A (RPA), replication factor C clamp loader (RFC) proteins (Rfc1, Rfc2, Rfc3, Rfc4, and Rfc5), RMI1 protein, ATR kinase, ATR-interacting protein (ATRIP), RecQ Helicase proteins (RECQL1, RECQL2, RECQL3, RECQL4 and RECQL5), Mismatch Repair (MMR) proteins (PMS2, PMS2, MLH1, MLH2, MLH3, MSH4, MSH5 and MSH6), Proliferating cell nuclear antigen (PCNA), DNA ligase, Flap endonuclease 1 (FEN1), anaphase promoting complex subunit 5, RecQ-mediated genome instability protein 1, Origin recognition complex subunit 1, Homeobox protein Meis2, DNA Topoisomerase III Alpha, DNA polymerase epsilon 4.

In another embodiment, the method further comprises modulating the function or level of expression of a protein that is involved in DNA replication, for example, the FANCM protein.

In another embodiment, the replication fork protein is selected from the group consisting of: RecQ Helicase proteins (RECQL1, RECQL2, RECQL3, RECQL4 and RECQL5), MMR proteins (PMS2, PMS2, MLH2, MLH3, MSH4, MSH5 and MSH6), and PCNA.

In another embodiment, the replication fork protein is a RecQ Helicase protein selected from the group consisting of: RECQL1, RECQL2, RECQL3, RECQL4 and RECQL5.

In another embodiment, the replication fork protein is an MMR protein selected from the group consisting of: PMS2, PMS2, MLH1, MLH2, MLH3, MSH4, MSH5 and MSH6.

In another embodiment, the replication fork protein is PCNA.

In another embodiment, the replication fork protein is a RecQ Helicase protein selected from the group consisting of: RECQL1, RECQL2, RECQL3, RECQL4 and RECQL5, and the replication fork modulator is emetine.

In another embodiment, the replication fork protein is a RecQ Helicase protein selected from the group consisting of: RECQL1, RECQL2, RECQL3, RECQL4 and RECQL5, and the replication fork modulator is siRNA.

In another embodiment, the replication fork protein is a RecQ Helicase protein selected from the group consisting of: RECQL1, RECQL2, RECQL3, RECQL4 and RECQL5, and the replication fork modulator is shRNA.

In another embodiment, the replication fork protein is an MMR protein selected from the group consisting of: PMS2, PMS2, MLH1, MLH2, MLH3, MSH4, MSH5 and MSH6, and the replication fork modulator is emetine.

In another embodiment, the replication fork protein is an MMR protein selected from the group consisting of: PMS2, PMS2, MLH1, MLH2, MLH3, MSH4, MSH5 and MSH6, and the replication fork modulator is siRNA.

In another embodiment, the replication fork protein is an MMR protein selected from the group consisting of: PMS2, PMS2, MLH1, MLH2, MLH3, MSH4, MSH5 and MSH6, and the replication fork modulator is shRNA.

In another embodiment, the replication fork protein is Proliferating cell nuclear antigen (PCNA) and the replication fork modulator is emetine.

In another embodiment, the replication fork protein is PCNA and the replication fork modulator is siRNA

In another embodiment, the replication fork protein is PCNA and the replication fork modulator is shRNA.

In another embodiment, the cell is selected from the group consisting of: pluripotent stem cell, induced pluripotent stem cell, and embryonic stem cell.

In another embodiment, the cell is a primate cell.

In another embodiment, the cell is a differentiated cell.

In another embodiment, the gene editing efficiency in the cell is greater than the gene editing efficiency in a cell that has not been contacted with a replication fork modulator.

The invention also provides for a method of editing a gene in a cell comprising contacting a cell with a replication fork modulator for a period of time before editing the gene in the cell, and editing the gene in the cell.

In one embodiment, the period of time is about 8 hours to about 7 days.

The invention also provides for a method of editing a gene in a cell comprising contacting a cell with a replication fork modulator during gene editing.

The invention also provides for a method of editing a gene in a cell comprising contacting a cell with a replication fork modulator for a period of time after editing the gene in the cell, and editing the gene in the cell.

In one embodiment, the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

In another embodiment, the replication fork modulator is a leading strand synthesis inhibitor.

In another embodiment, the replication fork modulator is a lagging strand synthesis inhibitor.

In another embodiment, the method further comprises contacting the cell with a gene editing vector.

In another embodiment, the gene editing vector is an adeno-associated virus (AAV) vector.

The invention also provides for a method of editing a gene in a cell of a subject, comprising: administering a gene editing vector to the subject; and administering a replication fork modulator to the subject.

In one embodiment, the replication fork modulator is administered after the gene editing vector is administered.

In another embodiment, the replication fork modulator is administered before the gene editing vector is administered.

In another embodiment, the gene editing vector and the replication fork synthesis modulator are administered at the same time.

In another embodiment, the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

In another embodiment, the replication fork modulator is a leading strand synthesis inhibitor.

In another embodiment, the replication fork modulator is a lagging strand synthesis inhibitor.

In another embodiment, the gene editing vector is an AAV vector.

The invention also provides for a method of editing a gene in a cell comprising: editing the gene in the cell; and contacting the gene edited cell with a replication fork modulator for a period of time.

In one embodiment, the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

In another embodiment, the replication fork modulator is a leading strand synthesis inhibitor.

In another embodiment, the replication fork modulator is a lagging strand synthesis inhibitor.

In another embodiment, the method further comprises contacting the cell with a gene editing vector.

In another embodiment, the gene editing vector is an AAV vector.

The invention also provides for a composition comprising a population of gene edited cells wherein the population of cells are obtained by modulating replication fork function in the cells with a replication fork modulator.

In one embodiment, the gene editing efficiency in the population of cells is greater than in a second population of cells gene edited in the absence of modulating replication fork function in the cells.

In one embodiment the method further comprises contacting the cells with a replication fork modulator.

In another embodiment, the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

In another embodiment, the replication fork modulator is a leading strand synthesis inhibitor.

In another embodiment, the replication fork modulator is a lagging strand synthesis inhibitor.

In another embodiment of the methods of the invention, the replication fork protein is selected from the group consisting of: RecQ Helicase proteins (RECQL1, RECQL2, RECQL3, RECQL4 and RECQL5), MMR proteins (PMS2, PMS2, MLH1, MLH2, MLH3, MSH4, MSH5 and MSH6), and PCNA.

In another embodiment of the methods of the invention, the replication fork protein is a RecQ Helicase protein selected from the group consisting of: RECQL1, RECQL2, RECQL3, RECQL4 and RECQL5.

In another embodiment of the methods of the invention, the replication fork protein is an MMR protein selected from the group consisting of: PMS2, PMS2, MLH1, MLH2, MLH3, MSH4, MSH5 and MSH6.

In another embodiment of the methods of the invention, the replication fork protein is PCNA.

In another embodiment of the methods of the invention, the replication fork protein is a RecQ Helicase protein selected from the group consisting of: RECQL1, RECQL2, RECQL3, RECQL4 and RECQL5, and the replication fork modulator is emetine.

In another embodiment of the methods of the invention, the replication fork protein is a RecQ Helicase protein selected from the group consisting of: RECQL1, RECQL2, RECQL3, RECQL4 and RECQL5, and the replication fork modulator is siRNA.

In another embodiment of the methods of the invention, the replication fork protein is a RecQ Helicase protein selected from the group consisting of: RECQL1, RECQL2, RECQL3, RECQL4 and RECQL5, and the replication fork modulator is shRNA.

In another embodiment of the methods of the invention, the replication fork protein is an MMR protein selected from the group consisting of: PMS2, PMS2, MLH1, MLH2, MLH3, MSH4, MSH5 and MSH6, and the replication fork modulator is emetine.

In another embodiment of the methods of the invention, the replication fork protein is an MMR protein selected from the group consisting of: PMS2, PMS2, MLH1, MLH2, MLH3, MSH4, MSH5 and MSH6, and the replication fork modulator is siRNA.

In another embodiment of the methods of the invention, the replication fork protein is an MMR protein selected from the group consisting of: PMS2, PMS2, MLH1, MLH2, MLH3, MSH4, MSH5 and MSH6, and the replication fork modulator is shRNA.

In another embodiment of the methods of the invention, the replication fork protein is PCNA and the replication fork modulator is emetine.

In another embodiment of the methods of the invention, the replication fork protein is PCNA and the replication fork modulator is siRNA

In another embodiment of the methods of the invention, the replication fork protein is PCNA and the replication fork modulator is shRNA.

In another embodiment of the methods of the invention, the cell is selected from the group consisting of: pluripotent stem cell, induced pluripotent stem cell, and embryonic stem cell.

In another embodiment of the methods of the invention, the cell is a primate cell.

In another embodiment, the cell is a differentiated cell.

In another embodiment, the methods comprise one or more replication fork modulators, for example, 1, 2, 3, 4, 5, 6, 7 or more.

In another embodiment, the methods of the invention comprise one or more replication fork modulators for example, emetine in combination with shRNA, or emetine in combination with siRNA, or emetine in combination with shRNA and siRNA or shRNA in combination with siRNA. In another embodiment, the methods comprise one or more replication fork modulators, for example a leading strand synthesis inhibitor in combination with shRNA, a leading strand synthesis inhibitor in combination with siRNA, a leading strand synthesis inhibitor in combination with shRNA and siRNA, or a leading strand synthesis inhibitor in combination with a lagging strand synthesis inhibitor. In another embodiment, the methods comprise one or more replication fork modulators, for example a lagging strand synthesis inhibitor in combination with shRNA, a lagging strand synthesis inhibitor in combination with siRNA or a lagging strand synthesis inhibitor in combination with shRNA and siRNA.

The invention also provides for a cell comprising a gene editing vector and an exogenous replication fork modulator, and a cell that is derived or differentiated therefrom.

In one embodiment, the gene editing vector is an AAV vector.

The invention also provides a cell comprising a gene modification and an exogenous replication fork modulator, and a cell that is derived or differentiated therefrom.

In one embodiment, the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

In another embodiment, the replication fork modulator is a leading strand synthesis activator or a leading strand synthesis inhibitor.

In another embodiment, the replication fork modulator is a lagging strand synthesis activator or a lagging strand synthesis inhibitor.

The invention also provides for a method of treating a disease in a subject in need comprising administering to the subject an effective amount of a cell of the invention.

The invention also provides for a method of transplantation in a subject in need comprising administering to the subject an effective amount of a cell of the invention.

### BRIEF DESCRIPTION OF THE DRAWINGS

The present invention is further described below with reference to the following non-limiting examples and with reference to the following figures.

FIG. 1 is a schematic of a human replication fork.

FIG. 2 is a graph demonstrating the effect of emetine on gene editing in mouse Hepa1-6 cells.

FIG. 3 is a graph demonstrating the effect of emetine on in vivo gene editing in mouse liver.

FIG. 4 is a graph demonstrating the effect of inhibition of RecQ helicase proteins by siRNAs on gene editing.

FIG. 5 is a graph demonstrating the effect of inhibition of RecQ helicase proteins by shRNAs on gene editing.

FIG. 6 is a graph demonstrating the effect of inhibition of RecQ helicase proteins by shRNAs on gene editing.

FIG. 7 is a graph demonstrating the effect of inhibition of mismatch repair (MMR) proteins by siRNAs on gene editing.

FIG. 8 is a graph demonstrating the effect of inhibition of mismatch repair (MMR) protein combinations by siRNAs on gene editing.

FIG. 9 is a graph demonstrating the effect of inhibition of PCNA by shRNAs on gene editing.

FIG. 10 presents vectors useful according to the invention ((A) shRNA vector, (B) AAV-mAlb-GFP, (C) AAV-mAlb-Luciferase), (D) AAV-HPe3 (AAV2-HPe3) and (E) AAV2-HSN5′ and MLV-LHSN63Δ530.

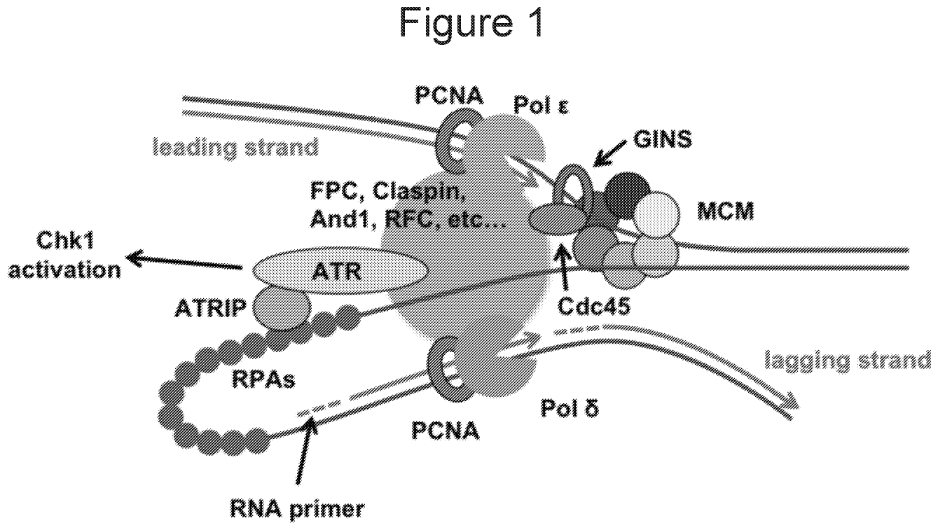

### DETAILED DESCRIPTION OF THE INVENTION

The present invention relates, at least in part, to a method of gene editing that comprises modulating the activity or expression of a protein associated with a replication fork, or a gene encoding a protein associated with a replication fork, or modulating gene editing, by the use of a replication fork modulator.

### Definitions

As used herein, “gene editing” or “genetic engineering” means modification of a target DNA sequence by insertion, deletion, substitution, replacement or alteration of one or more nucleotides, for example, to repair an undesired genetic mutation associated with a particular disease or disorder. Editing of a gene may result in a gene that is not expressed, is expressed at a level that is greater than or less than the level of expression of an unedited gene, fails to produce a wild type protein, produces a mutant form of a protein or is expressed at a different time or in a different environment as compared to an unedited gene. A gene editing vector may be used to edit a gene in a cell.

An “edited cell” is a cell in which an editing event has occurred. In an embodiment, an “edited cell” includes a cell that has been contacted with a gene editing vector and a replication fork modulator. In an embodiment, an “edited cell” includes a cell comprising a gene editing vector and a replication fork modulator. In another embodiment, an “edited cell” also includes a cell comprising a replication fork modulator or a gene editing vector. In a further embodiment, an “edited cell” is also a cell that is derived or differentiated from a cell in which an editing event has occurred. A cell may be gene edited by any method known in the art, for example, by introduction of an editing vector.

As used herein, “replication fork modulator” means an agent that modulates replication fork function, for example, DNA synthesis or unwinding of the DNA double helix. As used herein, “modulates” means increases or decreases. In an embodiment, a “replication fork modulator” includes an agent that modulates the level of activity or expression of a protein, or the corresponding gene, wherein the protein is associated with a replication fork. A replication fork modulator may increase or decrease the level of expression of a protein, or the corresponding gene, or the level of activity of a protein. In one embodiment, a replication fork modulator directly modulates a level of expression or activity. In another embodiment, a replication fork modulator indirectly modulates a level of expression or activity, for example, by directly modulating a protein which in turn directly modulates a level of expression or activity of a protein associated with a replication fork.

In an embodiment, the replication fork modulator modulates gene editing activity in a cell. In an embodiment, the replication fork modulator increases or decreases the amount of gene editing vector that enters a cell. In another embodiment, the replication fork modulator increases or decreases the stability of a gene editing vector in a cell. In another embodiment, the replication fork modulator increases or decreases the level of homologous pairing between the vector and the homologous chromosomal sequence at a target locus. In another embodiment, the replication fork modulator increases or decreases recombination between the vector and the target locus. In another embodiment, the replication fork modulator increases or decreases the DNA repair synthesis process wherein the vector sequence is copied into the opposite strand of the chromosome. In another embodiment, the replication fork modulator increases or decreases the formation of site specific double stranded breaks at the chromosomal target locus, in another embodiment, the replication fork modulator increases or decreases homology directed repair and/or non-homologous end joining at a double stranded break.

As used herein, “activate” or “increase”, as it refers to the level of expression or activity, means, increase, for example by 2-fold or more, for example, 2-fold, 5-fold, 10-fold, 20-fold, 50-fold, 100-fold or more, as compared to a control level of activity or expression. Activate or increase also means increase by 5% or more, for example, 5%, 10%, 15%, 20%, 25%, 30%, 35%, 40%, 45%, 50%, 55%, 60%, 65%, 70%, 75%, 80%, 85%, 90%, 9.5%, 99% or 100%, as compared to a control level of activity or expression. For example, the level of replication fork activity may be increased in the presence of a replication fork modulator compared to the level of replication fork activity in the absence of the replication fork modulator. In another example, the level of gene editing activity in a cell may be increased in the presence of a replication fork modulator compared to the level of gene editing activity in a cell in the absence of the replication fork modulator.

As used herein, “inhibit” or “decrease”, as it refers to the level of expression or activity means, reduce, for example by 2-fold or more, for example, 2-fold, 5-fold, 10-fold, 20-fold, 50-fold, 100-fold or more, as compared to a control level of activity or expression. Inhibit also means reduce by 5% or more, for example, 5%, 10%, 15% 20%, 25%, 30%, 35%, 40%, 45%, 50%, 55%, 60%, 65%, 70%, 75%, 80%, 85%, 90%, 95%, 99% or 100%, as compared to a control level of activity or expression. Inhibition also means complete inhibition such that no expression or activity is detectable. For example, the level of replication fork activity may be inhibited in the presence of a replication fork modulator compared to the level of replication fork activity in the absence of the replication fork modulator. In another example, the level of gene editing activity may be inhibited in the absence of the replication fork modulator compared to the level of gene editing activity in a cell in the presence of the replication fork modulator.

In certain embodiments, a replication fork modulator “specifically modulates” a level of expression or activity. A replication fork modulator that specifically modulates, increases or decreases a particular level of expression or an activity but does not significantly affect another level of expression or activity. In an embodiment, a replication fork modulator that specifically inhibits an activity associated with a replication fork, for example lagging strand synthesis, inhibits lagging strand synthesis but does not significantly affect leading strand synthesis. In other embodiments, a replication fork modulator that specifically increases an activity associated with a replication fork, for example lagging strand synthesis, increases lagging strand synthesis but does not significantly affect leading strand synthesis.

In certain embodiments, a replication fork modulator that “specifically modulates” the level of expression of a gene or protein associated with a replication fork, for example, DNA pol δ, modulates the level of DNA pol δ and does not significantly affect the level of expression of another gene or protein associated with the replication fork.

A replication fork modulator useful according to the invention includes hut is not limited to small molecules, proteins, peptides and nucleic acids, for example, an aptamer, small interfering RNA (siRNA), short hairpin RNA (shRNA), small internally segmented interfering RNA, microRNA, antisense oligonucleotide, and signal interfering DNA (siDNA). In certain embodiments, a replication fork modulator is a siRNA or a shRNA that is specific for a RecQ helicase protein, proliferating cell nuclear antigen or a mismatch repair protein. In one embodiment, a replication fork modulator is emetine, dehydroemetine, emetine dihydrochloro hydrate, cephaeline, or salts thereof. Additional replication fork modulators useful according to the invention include, but are not limited to, monoclonal antibodies directed to proteins associated with the replication fork that are involved in replication fork activity, bi-specific T-cell engagers, sequence-specific epigenetic regulators, for example, Cas9-based molecules linked to chromatin modifiers, targeted protein degradation systems (e.g., the ubiquitin system), and inducible regulated expression systems to control replication fork protein genes.

In an embodiment, a “composition” comprises a gene editing vector and/or a replication fork modulator. In a specific embodiment, a composition comprises a gene editing vector and a replication fork modulator. In another embodiment, a composition comprises a replication fork modulator or a cell comprising a gene editing vector. In some embodiments, a composition comprises an edited cell. In some embodiments a composition does not comprise an edited cell. In some embodiments, a composition comprises a cell derived or differentiated from an edited cell. A “composition” may include formulations comprising a composition of the invention.

The term “contacting” or “contact” as used herein in connection with contacting a cell with a composition of the invention, refers to any means by which the composition of the invention is brought into sufficient proximity and/or in direct contact with a cell. In some embodiments, contact refers to culturing a cell in a medium that comprises a composition of the invention. In another embodiment, contact refers to administering a composition of the invention to a subject.

As used herein, “population of cells” means two or more cell(s).

As used herein, “gene editing efficiency” means the level of gene editing, for example, the number of editing events in a cell, the frequency of editing events, the number of edited cells in a population of cells, or the speed at which editing occurs in a cell or a population of cells.

An increase in gene editing efficiency may be an increase of 2-fold or more, for example, 2-fold, 5-fold, 10-fold, 20-fold, 50-fold, 100-fold or more, as compared to a control level of gene editing. Increase also means increase by 5% or more, for example, 5%, 10%, 15% 20%, 25%, 30%, 35%, 40%, 45%, 50%, 55%, 60%, 65%, 70%, 75%, 80%, 85%, 90%, 95%, 99% or 100% as compared to a control level of gene editing.

In one embodiment, an increase in gene editing efficiency is increased as compared to the gene editing efficiency in a control cell that has not been contacted with a replication fork modulator and a gene editing vector, a cell that has been contacted with a gene editing vector but has not been contacted with a replication fork modulator, a cell that has been contacted with a replication fork modulator but has not been contacted with a gene editing vector or, as compared to a predetermined control level of gene editing.

By “subject” is meant an organism. In an embodiment, a subject is a donor or recipient of a composition of the invention, or a progeny derived or differentiated therefrom. “Subject” also refers to an organism in need of gene editing. “Subject” also refers to an organism to which the cells of the invention may be administered. “Subject” also refers to an organism to which a replication fork modulator and/or a gene editing vector are administered. A subject may be a mammal or a mammalian cell, including a human or human cell. The term “subject” includes all vertebrates, e.g., mammals, e.g., rodents, e.g., mice, and non-mammals, such as non-human primates, e.g., sheep, dog, cow, chickens, amphibians, reptiles, etc. A “subject” may be treated in accordance with the methods of the present invention or screened for pharmaceutical drug development purposes. A subject according to some embodiments of the present invention includes a patient, human or otherwise, in need of therapeutic or prophylactic treatment for a disease according to the invention, or who receives prophylactic or therapeutic treatment.

Various methodologies of the instant invention include steps that involve comparing a value, level, feature, characteristic, and/or property, to a “control. A “control” may be any control or standard familiar to one of ordinary skill in the art useful for comparison purposes. In one embodiment, a “control” is a value, level, feature, characteristic, property, etc. determined prior to performing a method of editing a gene in a cell, as described herein. For example, the occurrence of editing, the number of editing events, the efficiency of editing and the number of edited cells in a population of cells may be determined prior to introducing a replication fork modulator and/or a gene editing vector into a cell, or in the absence of a replication fork modulator and/or a gene editing vector. A “control” also includes a cell that has been edited in the absence of a replication fork modulator. In another embodiment, a “control” is a value, level, feature, characteristic, property, etc. determined in a cell or organism, e.g., a control or normal cell or organism, exhibiting, for example, normal traits. In yet another embodiment, a “control” is a predefined value, level, feature, characteristic, property, etc. determined prior to the addition of a replication fork modulator and/or a gene editing vector.

Accordingly, a “control subject” may refer to a subject to which a subject that has been treated according to the present invention is compared. The “control subject” may not be diagnosed with a disease or condition or treated for a disease or condition. A “control subject” may also refer to a subject that is not at risk of developing a disease or condition. A “control subject” may also refer to a subject to which a cell according to the invention has not been administered. A “control subject” may also refer to a subject that has not been treated with a replication fork modulator and/or an editing vector.

A “control cell” may refer to a cell to which a cell that has been contacted with a gene editing vector and/or a replication fork modulator is compared. The “control cell” may not have been contacted with a gene editing vector and/or a replication fork modulator. The “control cell” may have been contacted with a gene editing vector and/or a replication fork modulator under different conditions, including dosage, length of time etc., as compared to the cell for which it is a control.

The methods of the invention are used to provide cells in which an editing event has occurred as well as cells that are derived or differentiated from a cell in which an editing event has occurred. In certain embodiments, the edited cell comprises a replication fork modulator or a gene editing vector. The invention also provides for cells comprising a replication fork modulator and a gene editing vector. The methods provide for the production of cells having an increased frequency of gene editing events, and a population of cells having an increased number of edited cells. The methods also provide for a more efficient means of generating edited cells. The cells of the invention are useful for alleviation of symptoms, or treatment of a disease or transplantation.

### Gene Editing

A cell may be gene edited by any method known in the art, for example, by introduction of an editing vector. Gene editing vectors useful according to the invention include viral and non-viral vectors. Viral vectors include but are not limited to retrovirus. gammaretrovirus, lentivirus, herpes virus, adenovirus, adenoassociated viral (AAV) vectors and parvoviral vectors.

Non-viral vectors include but are not limited to plasmid vectors, for example, pORT, pCOR, pFAR, Minicircle plasmids, Minivector plasmids, Miniknot plasmid, and MIDGE, MiLV and ministring plasmids (See, Hardee et al. 2017, Genes, 8: 65). Additional non-viral vector systems/methods for introducing genetic material into a cell for gene editing include physical methods (needle administration, ballistic DNA, electroporation, sonoporation, photoporation, magnetofection, hydroporation, mechanical massage), chemical carriers (calcium phosphate, silica, gold, cationic lipids, lipid nano emulsions, solid lipid nanoparticle and peptide based delivery methods) and polymer based vectors (polyethylenimine, chitosan, poly (DL-lactide) and poly (DL-lactide-co-glycoside), dendrimers and polymethacrylate) (See, Ramamoorth et al. J. Clinical and Diagnostic Res., 2015, 9: 1-6). DNA, RNA and oligonucleotides are also useful for gene transfer.

CRISPR based methods are also useful for gene editing.

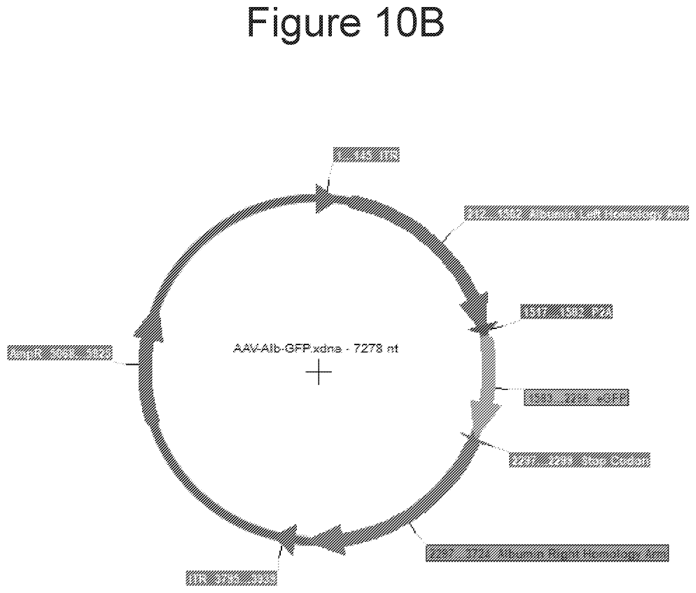

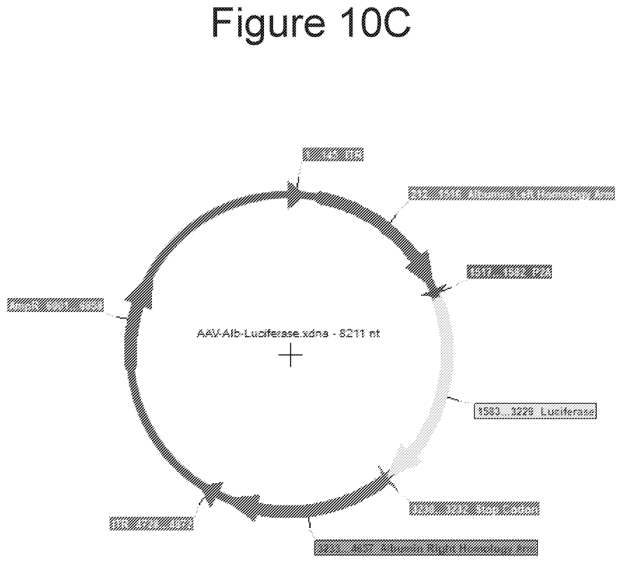

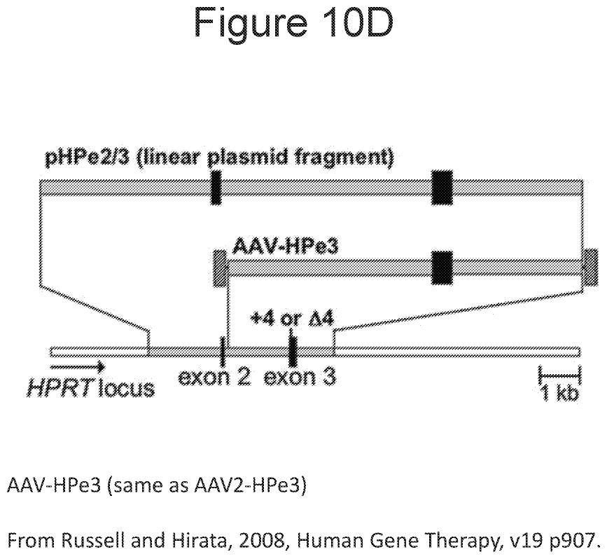

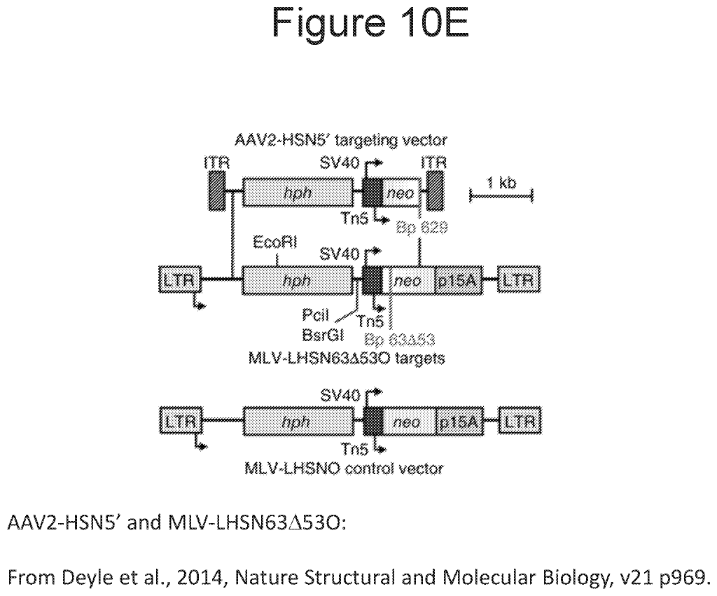

Gene editing may be performed using adeno-associated virus (AAV) vector gene targeting methods (See, Inoue et al., 1999, J. Virology, 73: 7376-7380 and Khan et al., 2011, Nature Protocols, 6: 482-501), for example, using AAV-mAlb-GFP, AAV-HPe3, AAV2-HSN5′, and AAV-Alb-Luciferase (see FIG. 10). Other AAV vectors useful according to the invention are known in the art.

The invention also provides for an in vivo method of editing a gene in a subject comprising administering to a subject a gene editing vector and a replication fork modulator of the invention.

An appropriate subject may be treated with a replication fork modulator and a gene editing vector either separately or simultaneously.

The invention provides for an in vivo method of editing a gene in a subject comprising administering to a subject a single composition comprising both a gene editing vector and a replication fork modulator. In an embodiment, the invention provides for an in vivo method of editing a gene in a subject comprising simultaneously administering a first composition comprising a gene editing vector and a second composition comprising a replication fork modulator.

The invention also provides for an in vivo method of editing a gene in a subject comprising sequentially administering to the subject a first composition comprising a gene editing vector and, separately administering a second composition comprising a replication fork modulator. In one embodiment a composition comprising a gene editing vector is administered before administration of a composition comprising a replication fork modulator. In another embodiment, a composition comprising a replication fork modulator is administered before administration of a composition comprising the gene editing vector. In an embodiment a composition comprising a gene editing vector can be administered and, after a period of time, a composition comprising a replication fork modulator is administered. In another embodiment, a composition comprising a replication fork modulator is administered and, after a period of time, a composition comprising a gene editing vector is administered.

As used herein, a “period of time” may be 15 minutes or more, for example, 15 minutes, 30 minutes, 1 hour, 2 hours, 3 hours, 4 hours, 5 hours, 6 hours, 7 hours, 8 hours to 24 hours, for example, 8, 9, 10, 11, 12, 13, 14, 15, 16, 17, 18, 19, 20, 21, 22, 23 and 24 hours or more. A period of time also includes 10-20 hours, 12-18 hours, 12-15 hours, 15 to 18 hours, 8-10 hours or 10-12 hours. In certain embodiments, a period of time is 2 days or more, for example, 2 days, 3 days, 4 days, 5 days, 6 days, 7 days, 8 days, 9 days, 10 days, 11 days, 12 days, 13 days, 14 days, 15 days, 20 days, 25 days, 26 days, 27 days, 28 days, 29 days, 30 days or 31 days or more.

The replication fork modulator and/or the gene editing vector may be administered once or multiple times. The replication fork modulator and the gene editing vector may be administered by any known method, for example as described below in the section entitled “Dosage and Mode of Administration”. In certain embodiments, the gene editing vector and/or the replication fork modulator are administered in a cell. In other embodiments, the gene editing vector and the replication fork modulator are not administered in a cell.

The methods of the invention may be used to edit any gene of interest.

The major histocompatibility complex (MHC) is a cell surface multi-component molecule found in all vertebrates that mediates interactions of leukocytes with other leukocytes or other cells. The MHC gene family is divided into three groups: class I, class II and class III. In humans, MHC is referred to as human leukocyte antigen (HLA). Class I molecules consist of an a, chain (or heavy chain) and β2 microglobulin (B2M), whereas the class II molecules consist of two homologous subunits: the alpha subunit and beta subunit.

In one embodiment, the methods of the invention may be used to edit a human leukocyte antigen (HLA) class I or class II-related gene. In another embodiment, the methods of the invention may be used to edit a B2M gene.

The HLA class I (HLA-I) protein is expressed on all nucleated cells and consists of an HLA class I heavy chain (or α chain) and B2M. HLA class I protein presents peptides on the cell surface to CD8+ cytotoxic T cells. Six HLA class I α chains have been identified to date, including three classical (HLA-A, HLA-B and HLA-C) and three non-classical (HLA-E, HLA-F and HLA-G) α chains. The specificity for peptide binding on the HLA class I molecule peptide binding cleft is determined by the α chain. Recognition by CD8+ T cells of the peptides presented by the HLA class I molecule mediates cellular immunity.

HLA class II molecules (HLA-II) are transmembrane proteins found only on professional antigen-presenting cells (APCs) including macrophages, dendritic cells and B cells. In addition, solid organs may sometimes express HLA class II genes that participate in immune rejection. HLA class II (HLA-II) molecules or proteins present on the cell surface peptide antigens from extracellular proteins including proteins of an extracellular pathogen, while HLA class I proteins present peptides from intracellular proteins or pathogens. Loaded HLA class II proteins on the cell surface interact with CD4+ helper T cells. The interaction leads to recruitment of phagocytes, local inflammation, and/or humoral responses through the activation of B cells. Several HLA class II gene loci have been identified to date, including HLA-DM (FILA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DRA α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

The HLA class I and/or class II proteins from an allogeneic source constitutes a foreign antigen in the context of transplantation. The recognition of non-self HLA class I and/or class II proteins is a major hurdle in using pluripotent cells for transplantation or replacement therapies. Cells of the invention comprising an edited HLA class I or class II-related gene, and/or a B2M gene, may be particularly useful for cell-based therapies.

In other embodiments, the methods of the invention may be used to edit any one of the following genes: RFXANK, RFXAP, RFX5, CIITA, CD1d, HPRT1 and albumin. The method of the invention may be used to edit any gene.

### Cells

A cell useful for editing according to the methods of the invention may be any cell, for example a mammalian cell. In certain embodiments, the method of editing a gene according to the invention is performed in a stern cell selected from the group consisting of a hematopoietic stem cell, an embryonic stem cell, a pluripotent stem cell, an induced pluripotent stem cell, a liver stem cell, a neural stem cell, a pancreatic stem cell and a mesenchymal stem cell. “A pluripotent stern cell” refers to a stern cell that has the potential to differentiate into any of the three germ layers: endoderm, mesoderm or ectoderm. “An adult stem cell,” is multipotent in that it can produce only a limited number of cell types. “An embryonic stem (ES) cell” refers to a pluripotent stem cell derived from the inner cell mass of the blastocyst, an early-stage embryo. “Induced pluripotent stem cells (iPS cells)” are pluripotent stem cells artificially derived from a non-pluripotent cell, typically an adult somatic cell, by artificially inducing expression of certain genes.

In another embodiment, the method of editing a gene according to the invention is performed in a differentiated cell including but not limited to a dendritic cell, a lymphocyte, a red blood cell, a platelet, a hematopoietic cell, a pancreatic islet cell, a liver cell, a muscle cell, a keratinocyte, a cardiomyocyte, a neuronal cell, a skeletal muscle cell, an ocular cell, a mesenchymal cell, a fibroblast, a lung cell, a gastrointestinal tract cell, a vascular cell, an endocrine cell, a skin cell, an adipocyte and a natural killer cell.

The invention therefore provides for a method of editing a gene in a stem cell or a differentiated cell. The invention also provides for an edited stem cell and progeny derived or differentiated therefrom, and an edited differentiated cell and progeny derived therefrom, including a cell comprising a replication fork modulator and/or a gene editing vector. A cell of the invention also includes a cell comprising a replication fork modulator and a gene editing vector that has not been edited prior to administration to a subject, but is edited after administration to the subject.

### Replication Fork Modulators

A replication fork modulator may modulate, either directly or indirectly, an activity that is associated with/occurs at a replication fork, for example, DNA synthesis or unwinding of the DNA double helix. A replication fork modulator may modulate the level of activity or expression of a protein associated with a replication fork or the corresponding gene encoding a protein associated with a replication fork.

A replication fork modulator useful according to the invention includes but is not limited to small molecules, proteins, peptides and nucleic acids, for example, small interfering RNA (siRNA), short hairpin RNA (shRNA), aptamer, small internally segmented interfering RNA, microRNA, antisense oligonucleotide, and signal interfering DNA (siDNA). In an embodiment, a replication fork modulator is a siRNA, aptamer, small internally segmented interfering RNA, microRNA, antisense oligonucleotide or a shRNA that is specific for a RecQ helicase protein(s), proliferating cell nuclear antigen or a mismatch repair protein(s). In another embodiment, a replication fork modulator is emetine, dehydroemetine, emetine dihydrochloro hydrate, cephaeline, or salts thereof. A replication fork modulator may specifically modulate DNA synthesis at a replication fork, for example, increase and/or decrease leading strand synthesis, or increase and/or decrease lagging strand synthesis.

In an embodiment, a replication fork modulator specifically increases leading strand synthesis. In another embodiment, a replication fork modulator specifically decreases leading strand synthesis. In another embodiment, a replication fork modulator specifically increases lagging strand synthesis. In another embodiment, a replication fork modulator specifically decreases lagging strand synthesis. In another embodiment, a replication fork modulator increases or decreases leading strand and lagging strand synthesis.

In an embodiment, a replication fork modulator specifically increases or specifically decreases leading strand synthesis, but does not significantly increase or decrease lagging strand synthesis. In another embodiment, a replication fork modulator specifically increases or specifically decreases lagging strand synthesis, but does not significantly increase or decrease leading strand synthesis. Leading and lagging strand synthesis at a replication fork is measured/detected according to methods well known in the art, for example, as described in Burhans et al., 1991, The EMBO Journal,10: 4351-4360 and Schauer et al., 2017, Bio. Protoc. 7: 1-23).

In certain embodiments, the replication fork modulator inhibits lagging strand synthesis at the replication fork. Agents useful for specific inhibition of lagging strand synthesis at a replication fork include but are not limited to emetine, dehydroemetine, emetine dihydrochloro hydrate, cephaeline, or salts thereof; or a siRNA, aptamer, small internally segmented interfering RNA, microRNA, antisense oligonucleotide or shRNA that is specific for a molecule that modulates lagging strand synthesis, for example, DNA polymerase δ (Pol δ) or DNA polymerase α, DNA polymerase β, DNA primase and DNA ligase.

In certain embodiments, the replication fork modulator inhibits leading strand synthesis at the replication fork. Agents useful for specific inhibition of leading strand synthesis at a replication fork include but are not limited to an siRNA or shRNA that is specific for a molecule that modulates leading strand synthesis, for example, DNA polymerase ε (Pol ε).

As used herein, “inhibit” or “decrease”, as it refers to synthesis of the lagging strand at a replication fork or as it refers to synthesis of the leading strand at a replication fork, means, reduce, for example by 2-fold or more, for example, 2-fold, 5-fold, 10-fold, 20-fold, 50-fold, 100-fold or more as compared to a control level of lagging strand synthesis or leading strand synthesis, respectively. Inhibit also means decrease by 5% or more, for example, 5%, 10%, 15%, 20%, 25%, 30%, 35% 40%, 45%, 50%, 55%, 60%, 65%, 70%, 75%, 80%, 83%, 90%, 95%, 99%, or 100% as compared to a control level of lagging strand synthesis or leading strand synthesis, respectively. Inhibition also means complete inhibition such that no detectable synthesis of the lagging strand is detectable.

As used herein, “activate” or “increase”, as it refers to synthesis of the lagging strand at a replication fork or as it refers to synthesis of the leading strand at a replication fork, means, increase, for example by 2-fold or more, for example, 2-fold, 3-fold, 10-fold, 20-fold, 50-fold, 100-fold or more as compared to a control level of lagging strand synthesis or leading strand synthesis, respectively. Activate or increase also means increase by 5% or more, for example, 5% or more, for example, 5%, 10%, 15%, 20%, 25%, 30%, 35%, 40%, 45%, 50%, 55%, 60%, 65%, 70%, 75%, 80%, 85%, 90%, 95%, 99%, or 100% as compared to a control level of lagging strand synthesis or leading strand synthesis, respectively.

In one embodiment, inhibition of lagging strand synthesis is inhibition as compared to the level of lagging strand synthesis in a control cell that has not been contacted with a lagging strand synthesis inhibitor, a cell prior to contacting with a lagging strand synthesis inhibitor or, as compared to a predetermined control level of lagging strand synthesis.

In one embodiment, inhibition of leading strand synthesis is inhibition as compared to the level of leading strand synthesis in a control cell that has not been contacted with a leading strand synthesis inhibitor, a cell prior to contacting with a leading strand synthesis inhibitor or, as compared to a predetermined control level of leading strand synthesis.

A replication fork modulator may modulate the activity or level of expression of a protein or a gene expressing a protein associated with replication fork function or activity, including but not limited to: DNA polymerase α, DNA primase, RNA primase, DNA polymerase α1, DNA polymerase ε, DNA polymerase ε4, DNA polymerase δ, fork protection complex (FPC) components Timeless, Tipin, Claspin and And1, Cdc45, MCM 2-7 (mini-chromosome maintenance) helicase 2-7 hexamer proteins (Mcm2, Mcm3, Mcm4, Mcm5, Mcm6 and Mcm7), go-ichi-ni-san (GINS) complex proteins (Sld5, Psf1, Psf2 and Psf3), replication protein A (RPA), replication factor C clamp loader (RFC) proteins (Rfc1, Rfc2, Rfc3, Rfc4, and Rfc5), RMI1 protein, ATR kinase, ATR-interacting protein (ATRIP), RecQ Helicase proteins (RECQL1, RECQL2, RECQL3, RECQL4 and RECQL5), Mismatch Repair (MMR) proteins (PMS2, PMS2, MLH1, MLH2, MLH3, MSH4, MSH5 and MSH6), and Proliferating cell nuclear antigen (PCNA), DNA ligase and Flap endonuclease 1 (Flap 1). In other embodiments, a replication fork modulator modulates the activity and or level of expression of anaphase promoting complex subunit 5, RecQ-mediated genome instability protein 1, Origin recognition complex subunit 1, Homeobox protein Meis2, DNA Topoisomerase III Alpha, DNA polymerase epsilon 4 and FANCM protein. Any one of these proteins may be involved in leading and/or lagging strand synthesis.

A protein that is associated with replication fork function includes any protein that increases or decreases a function of the replication fork or an activity, including but not limited to, DNA synthesis, primer synthesis, unwinding of the DNA helix, that occurs at the replication fork, or increases or decreases the level of expression of a protein, or a gene encoding a protein, that is associated with the replication fork or itself modulates replication fork function.

In one embodiment, a replication fork modulator directly modulates a level of expression or activity. In another embodiment, a replication fork modulator indirectly modulates a level of expression or activity, for example, by directly modulating a protein which in turn directly modulates a level of expression or activity of a protein associated with a replication fork.

A replication fork modulator may modulate gene editing activity in a cell. In an embodiment, the replication fork modulator increases or decreases the amount of gene editing vector that enters a cell. In another embodiment, the replication fork modulator increases or decreases the stability of a gene editing vector in a cell. In another embodiment, the replication fork modulator increases or decreases the level of homologous pairing between the vector and the homologous chromosomal sequence at a target locus. In another embodiment, the replication fork modulator increases or decreases recombination between the vector and the target locus. In another embodiment, the replication fork modulator increases or decreases the DNA repair synthesis process wherein the vector sequence is copied into the opposite strand of the chromosome. In another embodiment, the replication fork modulator increases or decreases the formation of site specific double stranded breaks at the chromosomal target locus, in another embodiment, the replication fork modulator increases or decreases homology directed repair and/or non-homologous end joining at a double stranded break.

In one embodiment, the replication fork protein is a RecQ Helicase protein selected from the group consisting of: RECQL1, RECQL2, RECQL3, RECQL4 and RECQL5, and the replication fork modulator inhibitor is one or more of emetine, siRNA corresponding to a gene encoding the RecQ Helicase protein, for example as provided in Table 2, or shRNA corresponding to the gene encoding the RecQ Helicase protein, for example as provided in Table 3.

In another embodiment, the replication fork protein is a Mismatch Repair (MMR) protein selected from the group consisting of: PMS2, PMS2, MLH1, MLH2, MLH3, MSH4, MSH5 and MSH6, and the replication fork modulator is one or more of emetine, siRNA corresponding to a gene encoding the Mismatch Repair protein, for example as provided in Table 4, or shRNA corresponding to the gene encoding the Mismatch Repair Protein.

In another embodiment, the replication fork protein is Proliferating cell nuclear antigen (PCNA) and the replication fork modulator is one or more of emetine, siRNA corresponding to the gene encoding PCNA, or shRNA corresponding to the gene encoding PCNA.

### Dosage and Mode of Administration

The invention provides for methods of treating a disease or condition and/or transplantation comprising administering to a subject a composition of the invention.

The term “administering,” as used herein, refers to any mode of transferring, delivering, introducing, or transporting a composition of the invention to a subject. Modes of administration include, but are not limited to, oral, topical, intravenous, intraperitoneal, intramuscular, subdermal, intradermal, intranasal, transcutaneous and subcutaneous administration. For example, a composition of the invention may be delivered to a vein, artery, capillary, heart, or tissue of a subject, as well as to a specific population, or sub-population, of cells. In an embodiment, administration means delivery of a gene editing vector and a replication fork modulator to a subject intraperitoneally. Administration of a composition of the invention may be assessed by adding tracking agents.

Administration of a composition of the invention may occur prior to the detection of a disease in a subject, or the manifestation of symptoms characteristic of the disease or disorder, such that the disease or disorder is prevented or, alternatively, delayed in its progression. In one embodiment, the cells are administered via a delivery device including without limitation a syringe or a catheter.

By “effective amount” or “therapeutically effective amount” is meant the amount of a composition of the invention sufficient to ameliorate the symptoms of a disease or condition, or cause gene editing in a cell. By “effective amount” or “therapeutically effective amount” is also meant the amount of a composition of the invention for inducing a therapeutic or prophylactic effect for use in therapy to treat a disease or condition according to the invention. By “effective amount” or “therapeutically effective amount” is also meant an amount sufficient to achieve or at least partially achieve the desired effect. The effective amount of a composition of the invention, the mode of administration and the treatment regimen, may be determined by one of skill in the art.

By “ameliorate” is meant decrease, suppress, attenuate, diminish, arrest, delay the onset, or stabilize the development or progression of a disease.

The therapeutically effective amount of a cell of the invention can range from the maximum number of cells that is safely received by the subject to the minimum number of cells necessary for treatment, including but not limited to a dosage of about 10,000 cells/kg, about 20,000 cells/kg, about 30,000 cells/kg, about 40,000 cells/kg, about 50,000 cells/kg, about 100,000 cells/kg, about 200,000 cells/kg, about 300,000 cells/kg, about 400,000 cells/kg, about 500,000 cells/kg, about 600,000 cells/kg, about 700,000 cells/kg, about 800,000 cells/kg, about 900,000 cells/kg, about 1.1×10⁶ cells/kg, about 1.2×10⁶ cells/kg, about 1.3×10⁶ cells/kg, about 1.4×10⁶ cells/kg, about 1.5×10⁶cells/kg, about 1.6×10⁶ cells/kg, about 1.7×10⁶ cells/kg, about 1.8×10⁶ cells/kg, about 1.9×10⁶ cells/kg, about 2.1×10⁶ cells/kg, about 2.1×10⁶ cells/kg, about 1.2×10⁶ cells/kg, about 2.3×10⁶ cells/kg, about 2.4×10⁶ cells/kg, about 2.5×10⁶ cells/kg, about 2.6×10⁶ cells/kg, about 2.7×10⁶ cells/kg, about 2.8×10⁶ cells/kg, about 2.9×10⁶ cells/kg, about 3×10⁶ cells/kg, about 3.1×10⁶ cells/kg, about 3.2×10⁶ cells/kg, about 3.3×10⁶ cells/kg, about 3.4×10⁶ cells/kg, about 3.5×10⁶ cells/kg, about 3.6×10⁶ cells/kg, about 3.7×10⁶ cells/kg, about 3.8×10⁶ cells/kg, about 3.9×10⁶ cells/kg, about 4×10⁶ cells/kg, about 4.1×10⁶ cells/kg, about 4.2×10⁶ cells/kg, about 4.3×10⁶ cells/kg, about 4.4×10⁶ cells/kg, about 4.5×10⁶ cells/kg, about 4.6×10⁶ cells/kg, about 4.7×10⁶ cells/kg, about 4.8×10⁶ cells/kg, about 4.9×10⁶ cells/kg, or about 5×10⁶ cells/kg. In embodiment, a therapeutically effective amount of a cell of the invention can range from 100 cells/kg to about 10¹¹ cells/kg, for example, 10,000 cells/kg to about 10¹¹ cells/kg, 100,000 cells/kg, to about 10¹¹ cells/kg, 500,000 cells/kg to about 10¹¹ cells/kg, 1×10⁶ cells/kg to about 10¹¹ cells/kg, 2×10⁶ cell/kg to about 10¹¹ cells/kg, 5×10⁶ cells/kg to about 10¹¹ cells/kg, 1×1.0⁷ cells/kg to about 10¹¹ cells/kg, 1×10⁸ cells/kg to about 10¹¹ cells/kg, a×10⁹ cells/kg to about 10¹¹ cells/kg or 1×10¹⁰ cells to about 10¹¹ cells/kg.

In an embodiment, a therapeutically effective amount of a cell of the invention ranges from about 50,000 cells/kg-150,000 cells/kg, for example, 50,000 cells/kg-100 cell/kg, 60,000 cells/kg-100, 000 cells/kg, 75,000 cells/kg-150,000 cells/kg, 90,000 cells/kg-150,000 cells/kg. In another embodiment, the dosage of cells to a subject may be a single dosage or a single dosage plus additional dosages. A subject is said to be treated for a disease, if following administration of the cells of the invention, or following administration of a replication fork modulator and/or a gene editing vector, one or more symptoms of the disease are decreased or eliminated.

In an embodiment, cells of the invention are administered to a subject to treat dry macular degeneration or Stargardt's macular dystrophy via retro orbital injection or surgical slit implantation.

### Pharmaceutical Compositions

The compositions of the invention may be administered alone or as a pharmaceutical composition comprising diluents and/or other components. A pharmaceutical composition useful for the invention may comprise the compositions of the invention and a physiologically compatible buffer and, in certain embodiments, one or more pharmaceutically or physiologically acceptable carriers, diluents or excipients, in which the compositions of the invention retain activity and in which the cells of the invention remain viable. Such compositions may comprise buffers such as neutral buffered saline, phosphate buffered saline and the like; carbohydrates such as glucose, mannose, sucrose or dextrans, mannitol; dextrose, water glycerol, ethanol, proteins; polypeptides or amino acids such as glycine; antioxidants; chelating agents such as EDTA or glutathione; adjuvants (e.g., aluminum hydroxide); and combinations thereof, and preservatives.

Compositions comprising cells of the invention may be kept in the solution or pharmaceutical composition for short term storage without losing viability. In one embodiment, the cells are frozen for long term storage without losing viability according to cryopreservation methods well-known in the art.

### Methods of Treatment and Transplantation

The present invention provides for both prophylactic and therapeutic methods of treating a subject at risk of a disease or disorder, treatable via administration of a composition of the invention.

“Treatment”, or “treating” as used herein, is defined as the administration of a composition of the invention to a subject to cure, heal, alleviate, relieve, alter, remedy, ameliorate, improve or affect a disease or disorder, or symptoms of the disease or disorder. The term “treatment” or “treating” is also used herein in the context of administering a composition of the invention prophylactically.

As used herein, “diagnosing” or “identifying a patient or subject having” refers to a process of determining if an individual is afflicted with a disease or ailment, for example a disease provided herein. Subjects at risk for the disease may be identified by, for example, any or a combination of diagnostic or prognostic assays known in the art. “Disease,” “disorder,” and “condition” are commonly recognized in the art and designate the presence of signs and/or symptoms in a subject that are generally recognized as abnormal and/or undesirable. Diseases or conditions may be diagnosed and categorized based on pathological changes.

In an embodiment, “disease” refers to any one of cancer, tumor growth, cancer of the colon, breast, bone, brain and others (e.g., osteosarcoma, neuroblastoma, colon adenocarcinoma), chronic myelogenous leukemia (CML), acute myeloid leukemia (AML), acute promyelocytic leukemia (APL), cardiac cancer (e.g., sarcoma, myxoma, rhabdomyoma, fibroma, lipoma and teratoma); lung cancer e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma)); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

“Disease” also includes any one of rheumatoid spondylitis; post ischemic perfusion injury; inflammatory bowel disease; chronic inflammatory pulmonary disease, eczema, asthma, ischemia/reperfusion injury, acute respiratory distress syndrome, infectious arthritis, progressive chronic arthritis, deforming arthritis, traumatic arthritis, gouty arthritis, Reiter's syndrome, acute synovitis and spondylitis, glomerulonephritis, hemolytic anemia, aplastic anemia, neutropenia, host versus graft disease, allograft rejection, chronic thyroiditis, Graves' disease, primary binary cirrhosis, contact dermatitis, skin sunburns, chronic renal insufficiency, Guillain-Barre syndrome, uveitis, otitis media, periodontal disease, pulmonary interstitial fibrosis, bronchitis, rhinitis, sinusitis, pneumoconiosis, pulmonary insufficiency syndrome, pulmonary emphysema, pulmonary fibrosis, silicosis, or chronic inflammatory pulmonary disease.

In a further embodiment, the term “disease” includes any one or more of the following autoimmune diseases or disorders: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, and interstitial lung fibrosis.

In another embodiment, disease refers to any one of Wilson's disease, spinocerebellar ataxia, prion disease, Parkinson's disease, Huntington's disease, amytrophic lateral sclerosis, amyloidosis, Alzheimer's disease, Alexander's disease, alcoholic liver disease, cystic fibrosis, Pick's Disease, spinal muscular dystrophy or Lewy body dementia. In one aspect, the invention provides a method for preventing in a subject, a disease or disorder as described above by administering to the subject a composition of the invention.

Another aspect of the invention pertains to methods of treating subjects, by administering a composition of the invention to the subject.

Compositions of the invention may be tested in an appropriate animal model. For example, a composition of the invention may be used in an animal model to determine the efficacy, toxicity, or side effects of treatment with the composition. Alternatively, a composition of the invention (e.g., a gene editing vector and a replication fork modulator, for example, emetine) may be used in an animal model to determine the mechanism of action of such composition.

### Kits

The present invention provides for kits. In an embodiment, the kit comprises a carrier means, a gene editing vector and a replication fork modulator, for example emetine. In another embodiment, a kit comprises a carrier means and a gene editing vector or a replication fork modulator. A gene editing vector and/or a replication fork modulator may be provided in a cell, either in separate cells or together in a single cell. If desired, the kit is provided with instructions for using the kit to produce an edited cell. In another embodiment, a kit includes a cell of the invention, or a derivative or differentiated cell derived therefrom, and an appropriate culture medium suitable for growth and maintenance of the cell. In another embodiment, a kit comprises a cell of the invention, or a derivative or differentiated cell derived therefrom, comprising a gene editing vector and, as a second component, a replication fork modulator. In another embodiment, a kit comprises a cell of the invention, or a derivative or differentiated cell derived therefrom, comprising a replication fork modulator and, separately, a gene editing vector. In another embodiment a kit comprises a cell having a gene editing vector. In another embodiment, a kit comprises a cell having a replication fork modulator. In another embodiment, a kit comprises a cell having both a gene editing vector and a replication fork modulator.

The carrier means may comprise any one of a box, carton, tube or the like, having in dose confinement therein one or more container means, such as vials, tubes, ampules, bottles and the like.

In other embodiments, the instructions include at least one of the following: description of the compositions; warnings; indications; counter-indications; animal study data; clinical study data; and/or references and may include instructions that generally include information about the use of the cells, gene editing vector and replication fork modulator for treating a subject having a disease or editing a cell in a subject. The instructions may be printed directly on the container (when present), as a label applied to the container, or as a separate sheet, pamphlet, card, or folder supplied in or with the container. The kit may also is include a compound that enhances the effect of the gene editing vector and/or the replication fork modulator. For example, a kit may comprises more than one replication fork modulator. The kit may also include a compound that contributes to the treatment of the subject, for example, an immunosuppressant.

### Animal Models

The edited cells of the invention are also applicable to animals, and may also be used to facilitate biomedical research of disease in a variety of animal model systems.

### Uses

The methods of the invention provide for in vitro and in vivo methods of production of edited cells that may be used for clinical applications including disease treatment and prevention. The cells of the invention and their differentiated progeny may also be used to identify compounds with a particular function, for example, treatment or prevention of disease, determine the activity of a compound of interest and or determine the toxicity of a compound of interest. Further, the present invention provides a cell therapy comprising transplanting edited cells or cells differentiated from an edited cell, into a patient.

In addition, the present invention provides a method for evaluation of physiological effect or toxicity of a compound, a drug, or a toxic agent, with use of various edited cells.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods in Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Dania rerio) (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The present invention is described by reference to the following Examples, which are offered by way of illustration and are not intended to limit the invention in any manner.

### Example 1

### Emetine Increases Gene Editing in Human HT-1080 Cells

The murine leukemia virus (MLV) vector LHSN63Δ53O contains a nonfunctional neomycin phosphotransferase target gene (neo) with a 53-bp deletion at nucleotide 63 of its open reading frame, a hygromycin phosphotransferase gene (hph) for selection, and a plasmid replication origin for recovering the target sites.

In some embodiments, a cell is gene edited using the HT-1080 (human fibrosarcoma cells) gene editing assay. This method uses HT-1080 neo/HPRT editing cells transduced with LHSN63Δ53O. HT-1080 cells contain an integrated copy of an MLV LHSN63Δ530 targeting locus and a 4 by deletion in the single copy, X-linked HPRT gene at exon 3. The cells are transduced with AAV2-HSN5′ and cultured in G418 to identify neo gene-edited cells. The AAV2-HSN5′ gene targeting vector contains sequences homologous to MLV-LHSN63Δ53O with a truncated neo gene that lacks the 53 by deletion. Alternatively, HT-1080 cells are transduced with AAV2-HPe3 targeting vector and selected with HAT medium to identify HPRT gene-edited cells (See Russell et al., 2008, Human Gene Therapy, 19: 907-914 and Deyle et al., 2014, Nature Structural & Molecular Biol., 21: 969-975). Targeted clones are expanded as a polyclonal population. Untargeted control cells are generated by transducing HT-1080 cells with the MLV vector LHSNO, which is identical to LHSN63Δ53O except that it contains a functional neo gene.

In another embodiment, a cell is gene edited using an HT-1080 GFP mutant editing line and the AAVDJ-mAlb-GFP vector. Gene editing in the presence of this vector results in an albumin knock-in with resulting expression of green fluorescent protein (GFP). GFP positive cells may be detected by flow cytometry.

To determine the effect of emetine on gene editing, experiments were performed using human HT-1080 cells with LHSN63Δ530 provirus and HPRT exon 3Δ4, infected with AAV2-HSN5′ or AAV2-HPe3. Replicate samples of cells were treated under the following conditions:
AAV (MOI 2×10⁴) alone for 24 hours;
0.125 μM emetine for 8 hours followed by the addition of AAV (MOI 2×10⁴) and incubation for 24 hours;
0.25 μM emetine for 24 hours followed by the addition of AAV (MOI 2×10⁴) and incubation for 24 hours;
0.25 μM emetine for 24 hours followed by the addition of AAV (MOI 2×10⁴) and incubation with AAV for 24 hours;
0.25 μM emetine in the presence of AAV (MOI 2×10⁴) for 24 hours;
AAV (MOI-2×10⁴) for 8 hours followed by the addition of 0.25 μM emetine and incubation with emetine for 23 hours;
AAV (MOI-2×10⁴) for 24 hours followed by the addition of 0.25 μM emetine and incubation with emetine for 8 hours;
AAV (MOI-2×10⁴) for 8 hours followed by the addition of emetine (0.25 μM) and incubation with emetine for 18 hours, removal of emetine, followed by addition of a second dosage of emetine (0.25 μM) and incubation for 16 hours; or
AAV (MOI 2×10⁴) for 24 hours followed by the addition of emetine (0.25 μM) and incubation for 7 hours, removal of emetine, followed by addition of a second dosage of emetine (0.25 μM) for 2 hours.

For each experiment, 2×10⁴ cells were seeded and cultured in the presence of 4×10⁸ AAV. All experiments were performed in DMEM+10% FBS+pen/strep. Emetine dihydrochloride hydrate: E2375 (Sigma) was prepared in water and sterilized by filtration on 0.22 uM filter.

The results of the experiments are provided in Table 1 below. These data demonstrate that there is an increase in gene editing in the presence of emetine.

TABLE 1

| | Fold increase in editing | |
|---|---|---|
| | frequency due to emetine | |
| HT-1080 cells with LHSN63D530 | % G4158R emetine/ | % HATR/% |
| provirus and HPRT exon3D4 infected | % G418R no | HAR no |
| with AAV2-HSN5′ or AAV2-HPe3 | emetine | emetine |
| No emetine, just AAV for 24 hrs | 1.00 | 1.00 |
| 0.125 μM emetine for 8 hrs before | 2.40 | 8.39 |
| adding AAV | | |
| 0.25 μM emetine for 24 hrs before | 3.82 | 7.25 |
| adding AAV | | |
| 0.25 μM emetine for 24 hrs before | 6.64 | 11.19 |
| adding AAV + 24 hrs with AAV | | |
| 0.25 μM emetine for 24 hrs with AAV | 3.70 | 8.39 |
| AAV for 8 hrs, then added 0.25 μM | 1.96 | 1.99 |
| emetine for 23 hrs | | |
| AAV for 24 hrs then with 0.25 μM | 1.94 | 1.63 |
| emetine for 8 hrs | | |
| AAV for 8 hrs, then added emetine for | 8.47 | 2.88 |
| 18 hrs, then removed emetine for 8 | | |
| hrs, then added emetine for 16 hrs | | |
| AAV for 24 hrs, then emetine for 7 | 2.63 | 1.56 |
| hrs, then removed emetine for 16 hrs, | | |
| then added emetine for 2 hrs | | |

### Example 2

### Emetine Increases Gene Editing in Mouse Hepa1-6 Cells

To determine the effect of emetine on gene editing, experiments were performed using mouse Hepa1-6 hepatoma cells using the vector AAVDJ-mAlb-GFP. Gene editing in the presence of this vector results in an albumin knock-in with resulting expression of green fluorescent protein (GFP).

On day 0, 100,000 cells were plated in 1.5 ml DMEM with 10% FBS, 100 units/mL penicillin and 100 μg/mL streptomycin. On day 1, cells were transduced with AAVDJ-mAlb-GFP (multiplicity of (MOI)—50,000) by removing the media and adding to the cells 1.5 ml of fresh media (DMEM with 10% FBS, 100 units/mL penicillin, 100 μg/mL streptomycin) containing the desired MOI of the AAVDJ-mAlb-GFP virus. For 100,000 cells, to achieve an MOI of 50,000, 5×10⁶ vector genomes were added.

On day 2, fresh medium (1.5 ml of DMEM with 10% FBS, 100 units/mL penicillin, 100 μg/mL streptomycin) with or without emetine, was added. Replicate samples were treated with no emetine, or with 30 nM emetine, 100 nM emetine or 1000 nM emetine. A control sample lacked AAV. On day 3, the media was removed and 1.5 ml of DMEM with 10% FBS, 100 units/mL penicillin, 100 μg/mL streptomycin was added to the cells. Expression of GFP was measured on days 6, 7 and 10.

Flow cytometry was used to measure GFP expression. On the day of the assay (days 6, 7 and 10) cells were trypsinized and the resulting cell pellet was resuspended in 500 μL of FACS buffer (DPBS (Dulbecco's Phosphate Buffered Saline) without Ca2+, Mg2+, and supplemented with 2% FBS, 1 mM EDTA). The resulting cell suspension was analyzed for GFP positive cells on the BD FACScanto II. For samples treated with no AAV, 0 nM emetine, 30 nM emetine, and 100 nM emetine, 100,000 cells were analyzed, and for samples treated with 1000 nM emetine, 70,000 cells were analyzed.

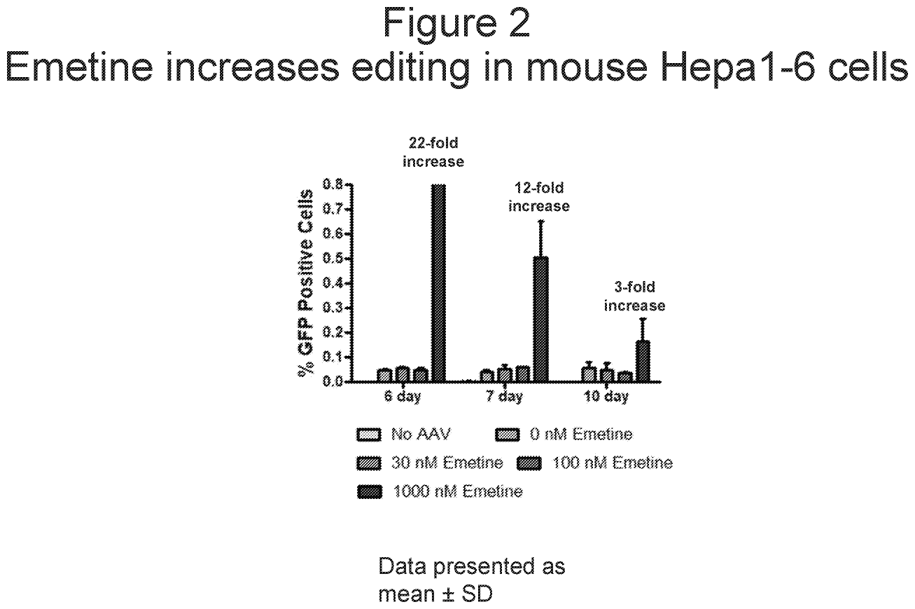

As demonstrated in FIG. 2, there was an increase in expression of GFP in the presence of 1000 nM emetine (22-fold at day 6, 12-fold at day 7 and 3-fold at day 10). The results demonstrate that emetine increases gene editing in a mouse hepatoma cell line.

### Example 3

### Emetine Increases In Vivo Editing of Mouse Liver

To determine the effect of emetine on gene editing in vivo, experiments were performed using C57BL/6 mice.

10 mice were utilized. On day 0, 5 mice (Group I), were given AAVDj-mALB-luciferase vector at an amount of 3×10¹¹ vg/mouse by retro orbital injection. Gene editing of the AAVDj-mALB-luciferase vector results in an albumin locus knock-in and luciferase expression. 5 mice (Group 2) were injected with AAVDj-mALB-luciferase vector, at an amount of 3×10¹¹ vg/mouse by retro orbital injection, and given emetine in DPBS, at a concentration of 5 mg/kg, by intraperitoneal injection. 5 mice (Group 3) were injected only with emetine at a concentration of 5 mg/kg, by intraperitoneal injection.

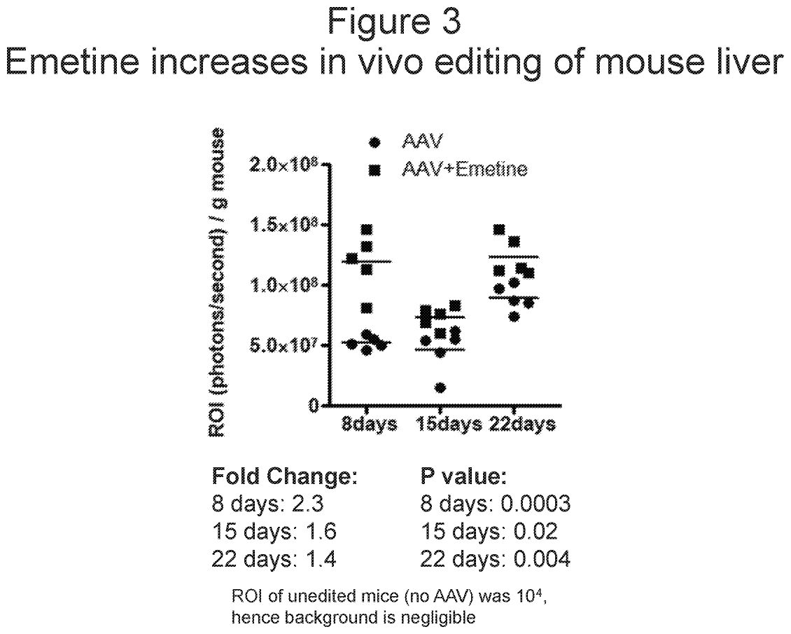

Emetine was administered to Groups 2 and 3 on days 1-5. On days 8, 11 and 22, in vivo imaging in the presence of 15 mg/kg D-luciferin, administered by intraperitoneal injection, was performed to determine the occurrence of editing. Following D-luciferin administration, the mice were anesthetized with isoflurane and placed in an IVIS imager and the peak bioluminescence was recorded (Gornalusse et al., 2017, Nature Biotech, 35(8): 765-772). As demonstrated in FIG. 3, emetine increases the level of gene editing in vivo in mouse liver. A 2.3 fold increase was observed at day 8, as compared to mice treated with AAV alone. At days 15 and 22, a 1.6 and 1.4 fold increase, respectively, were observed.

### Example 4

### Treatment of a Subject by Administration of an Edited Cell

In an embodiment, a subject is treated by administration of an edited cell prepared according to the methods described herein, for example, Examples 1 and 2.

An edited cell of the invention is used to treat a variety of diseases or conditions of a subject. The dosage, route of delivery and excipient may be modified according to the disease or condition being treated. In one embodiment, a retinal pigmented epithelial cell (RPE) derived from an edited cell of the invention is used to treat dry macular degeneration or Stargardt's macular dystrophy. On day 0, patients are treated with 50,000 cells; 100,000 cells, 150,000 cells, 500,000 cells or greater in any one of a number of physiological suitable buffers via retro orbital injection or surgical slit implantation. Patients are monitored for safety by assaying for: any grade 2 (NCI grading system) or greater adverse event related to the cell product; any evidence that the cells are contaminated with an infectious agent; and any evidence that the cells show tumorigenic potential. Successful engraftment and function of the cells is monitored by routine tests known in the art including; obtaining structural evidence that the cells have been implanted at the correct location (OCT imaging, fluorescein angiography, autofluorescence photography, slit-lamp examination with fundus photography); and electroretinographic evidence (mfERG) showing enhanced activity in the implant location and improvements in visual acuity.

### Example 5

Inhibition of RecQ Helicase by siRNA Increases Editing

To determine the effect of siRNA corresponding to the RecQ Helicase genes encoding the RecQ helicase proteins RECQL1, RECQL2, RECQL3, RECQL4 and RECQL5, on gene editing, experiments were performed using the HT-1080 neo/HPRT editing line. Each helicase gene was treated with a siRNA specific for each of RECQL1, RECQL2, RECQL3, RECQL4 and RECQL5.

Each helicase gene was treated with three different siRNA pairs (RECQL1 253/254, RECQL1 255/256, RECQL1 257/258, RECQL2 BLM 259/260, RECQL2 BLM 261/262, RECQL2 BLM 263/264, RECQL3 WRN 265/266, RECQL3 WRN 267/268, RECQL3 WRN 269/270, RECQL4 271/272, RECQL4 273/274, RECQL4 275/276, RECQL4 277/278, RECQL5 279/2806 and RECQL4 281/282).

TABLE 2

| siRNA | Sequence (5′ to 3′) |
|---|---|
| RECQL1 253 | CTCACTGAGTGATACTTTA (SEQ ID NO: 1) |
| RECQL1 254 | TAAAGTATCACTCAGTGAG (SEQ ID NO: 2) |
| RECQL1 255 | CAATCTGGTTCTAAGAATA (SEQ ID NO: 3) |
| RECQL1 256 | TATTCTTAGAACCAGATTG (SEQ ID NO: 4) |
| RECQL1 257 | GTCAGTGTTGTATTGGAAA (SEQ ID NO: 5) |
| RECQL1 258 | TTTCCAATACAACACTGAC (SEQ ID NO: 6) |
| RECQL2 BLM 259 | AGCAGCGATGTGATTTGC (SEQ ID NO: 7) |
| RECQL2 BLM 260 | GCAAATCACATCGCTGCT (SEQ ID NO: 8) |
| RECQL2 BLM 261 | ACCTTCCTATGATATTGAT (SEQ ID NO: 9) |
| RECQL2 BLM 262 | ATCAATATCATAGGAAGGT (SEQ ID NO: 10) |
| RECQL2 BLM 263 | GTGTCCATTACTTCAATAT (SEQ ID NO: 11) |
| RECQL2 BLM 264 | ATATTGAAGTAATGGACAC (SEQ ID NO: 12) |
| RECQL3 WRN 265 | GGATGAATGTGCAGAATAA (SEQ ID NO: 13) |
| RECQL3 WRN 266 | TTATTCTGCACATTCATCC (SEQ ID NO: 14) |
| RECQL3 WRN 267 | TTAACAGTCTGGTTAAACA (SEQ ID NO: 15) |
| RECQL3 WRN 268 | TGTTTAACCAGACTGTTAA (SEQ ID NO: 16) |
| RECQL3 WRN 269 | GACTTCATCTGCAGAGAGA (SEQ ID NO: 17) |
| RECQL3 WRN 270 | TCTCTCTGCAGATGAAGTC (SEQ ID NO: 18) |
| RECQL4 271 | GGCTCAACATGAAGCAGAA (SEQ ID NO: 19) |
| RECQL4 272 | TTTTGCTTCATGTTGAGCC (SEQ ID NO: 20) |
| RECQL4 273 | GCCACTGGTTCCTTCACCA (SEQ ID NO: 21) |
| RECQL4 274 | TGGTGAAGGAACCAGTGGC (SEQ ID NO: 22) |
| RECQL4 275 | GGGAATCTGTCCTGCAGAA (SEQ ID NO: 23) |
| RECQL4 276 | TTCTGCAGGACAGATTCCC (SEQ ID NO: 24) |
| RECQL5 277 | GGATGAAGCTCATTGTGTT (SEQ ID NO: 25) |
| RECQL5 278 | AACACAATGAGCTTCATCC (SEQ ID NO: 26) |
| RECQL5 279 | GTGTTGCGCTGCCTCTTGA (SEQ ID NO: 27) |
| RECQL5 280 | TCAAGAGGCAGCGCAACAC (SEQ ID NO: 28) |
| RECQL5 281 | TCTATGATGTGCAATTCAA (SEQ ID NO: 29) |
| RECQL5 282 | TTGAATTGCACATCATAGA (SEQ ID NO: 30) |
| Scrambled | ATTGACCGAATCACTCGTG (SEQ ID NO: 31) |
| siRNA control | |
| Scrambled | CACGAGTGATTCGGTCAAT (SEQ ID NO: 32) |
| siRNA control | |

HPRT and neo editing frequencies were measured by G418 or HAT selection, and normalized to a scrambled siRNA control.

10⁵ HT-1080 cells were plated in a 6-well plate on day 1. All experiments were carried out in triplicate. On day 2, cells were transduced with AAV-HSN or AAV-HPe3 at a MOI of 10⁴. 3 nM siRNA mixed with lipofectamine RNAiMAX reagent was then added to the cells. On day 4, the cells were treated with trypsin, and 0.1% of the cells were replated without selection in 6-well plates to determine the number of colony forming units. The remainder of the cells were replated in 6-well plates for further analysis of gene editing frequency.

On day 5, neo gene correction experiments were performed by adding G418 (700 ug/ml) to the experimental plates. On day 15, plates were washed with PBS and the colonies were stained with Coomassie brilliant blue G. The percentage of neo gene correction was calculated as the number of G418-resistant CFU per total CFU for each original well.

On day 5, HPRT gene correction experiments were performed by adding 1× HAT (hypoxanthine-aminopterin-thymidine medium) to the experimental plates. On day 15, plates were washed with PBS and the colonies were stained with Coomassie brilliant blue G. The percentage of HPRT gene correction was calculated as the number of HAT-resistant CFU per total CFU for each original well,

The CFU were normalized to the CFU of the control (scrambled siRNA) sample. The editing index was calculated by multiplying the normalized CFU by the mean of the percentage of neo gene correction.

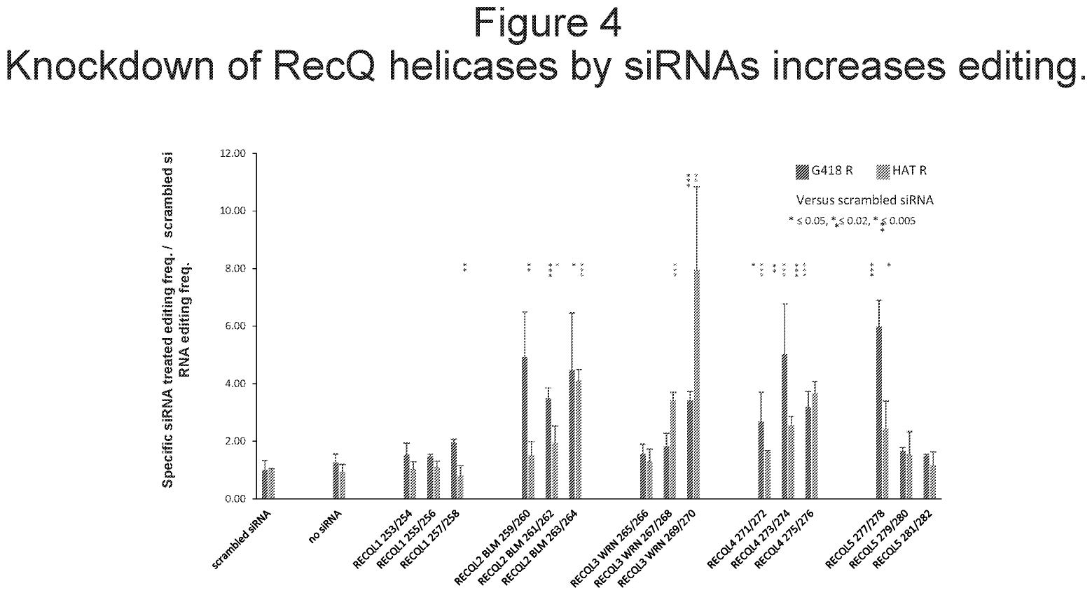

As demonstrated in FIG. 4, inhibition of RecQ helicases by siRNAs increases gene editing.

### Example 6

Inhibition of RecQ Helicase by shRNA Increases Editing

To determine the effect of shRNAs corresponding to the RecQ Helicase genes on gene editing, experiments were performed using the HT-1080 neo/HPRT editing line. Each helicase gene was treated with lentivirus vectors expressing a shRNA specific for each of RECQL1, RECQL2, RECQL3, RECQL4 and RECQL5 (sequences provided below in Table 3). A map of the shRNA vector is provided in FIG. 10. HPRT and neo editing frequencies were measured by G418 or HAT selection, and normalized to a scrambled shRNA control.

2×10⁴ HT-1080 cells were plated in 6-well plate on day 1. All experiments were carried out in triplicate. On day 2, cells were transduced with AAV-shRNA at an MOI of 2×10⁴. On day 3, cells were transduced with AAV-HSN or AAV-HPe3 at an MOI of 2×10⁴. On day 5, the cells were treated with trypsin and 0.1% of the cells were replated without selection in 6-well plates to determine the number of colony forming units. The remainder of the cells were replated in 6-well plates for further analysis of gene editing frequency.

On day 6, neo gene correction experiments were performed by adding G418 (700 ug/ml) to the experimental plates. On day 16, plates were washed with PBS and the colonies were stained with Coomassie brilliant blue G. The percentage of neo gene correction was calculated as the number of G418-resistant CFU per total CFU for each original well.

On day 6, HPRT gene correction experiments were performed by adding 1× HAT (hypoxanthine-aminopterin-thymidine medium) to the experimental plates. On day 16, plates were washed with PBS and the colonies were stained with Coomassie brilliant blue G. The percentage of HPRT gene correction was calculated as the number of HAT-resistant CFU per total CFU for each original well.

The CFU were normalized to the CFU of the control (scrambled siRNA) sample. The editing index was calculated by multiplying the normalized CFU by the mean of the percentage of neo gene correction.

TABLE 3

| shRNA | Sequence (5' to 3') |
|---|---|
| RECQL1 | TCAGTGTTGTATTGGAAATTGGATCCAATTTCCAATACAA |
| | CACTGACTTTTTCTCGAG (SEQ ID NO: 33) |
| RECQLI | GGCCCTCGAGAAAAAGTCAGTGTTGTATTGGAAATTGGAT |
| | CCAATTTCCAATACAACACTGA (SEQ ID NO: 34) |
| RECQL2 | TGTCCATTACTTCAATATTTGGATCCAAATATTGAAGTAA |
| | TGGACACTTTTTCTCGAG (SEQ ID NO: 35) |
| RECQL2 | GGCTCTCTCGAGAAAAAGTGTCCATTACTTCAATATTTGG |
| | ATCCAAATATTGAAGTAATGGACA (SEQ ID NO: 36) |
| RECQL3 | ACTTCATCTGCAGAGAGATTGGATCCAATCTCTCTGCAGA |
| | TGAAGTCTTTTTCTCGAG (SEQ ID NO: 37) |
| RECQL3 | GGCCCTCGAGAAAAAGACTTCATCTGCAGAGAGATTGGAT |
| | CCAATCTCTCTGCAGATGAAGT (SEQ ID NO: 38) |
| RECQL4 | CCACTGGTTCCTTCACCATTGATCCAATGGTGAAGGAACC |
| | AGTGGCTTTTTCTCGAG (SEQ ID NO: 39) |
| RECQL4 | GGCCCTCGAGAAAAAGCCACTGGTTCCTTCACCATTGGAT |
| | CCAATGGTGAAGGAACCAGTGG (SEQ ID NO: 40) |
| RECQL5 | GATGAAGCTCATTGTGTTTTGGATCCAAAACACAATGAGC |
| | TTCATCCTTTTTCTCGAG (SEQ ID NO: 41) |
| RECQL5 | GGCCCTCGAGAAAAAGGATGAAGCTCATTGTGTTTTGGAT |
| | CCAAAACACAATGAGCTTCATCC (SEQ ID NO: 42) |

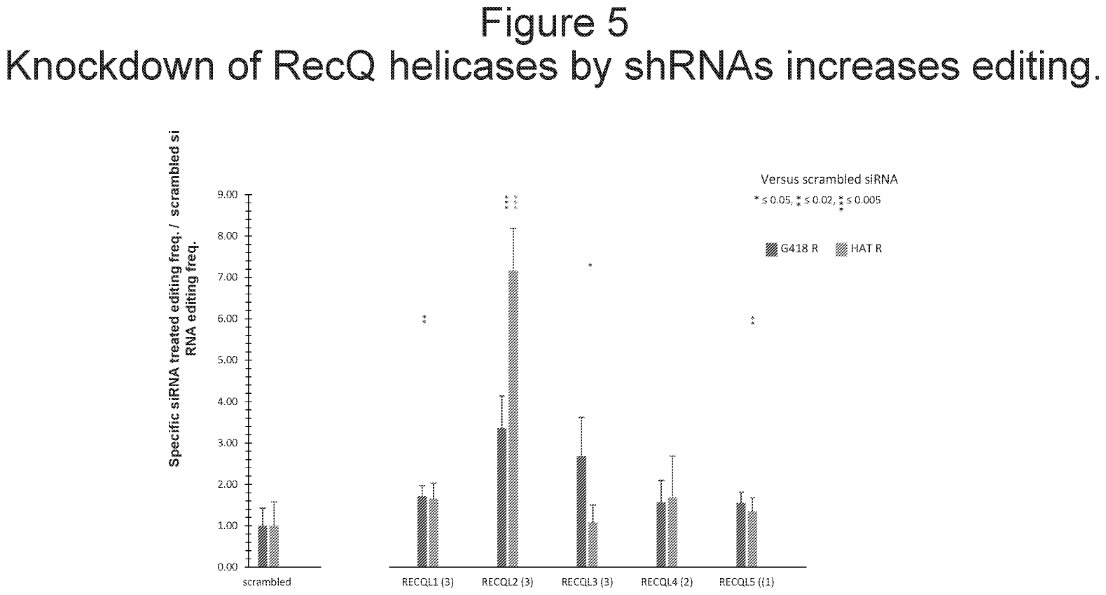

As demonstrated in FIG. 5, inhibition of RecQ helicases by shRNAs increases gene editing.

### Example 7

Inhibition of RecQ Helicase by shRNA Increases Editing

To determine the effect of shRNAs corresponding to the RecQ Helicase genes on gene editing, experiments were performed using the HT-1080 GFP mutant editing line. Each helicase gene was treated with a viral vector expressing a shRNA specific for each of RECQL1 (AAV-294-RECQL1), RECQL2 (AAV-295-RECQL2), RECQL3 (AAV-296-RECQL3), RECQL4 (AAV-297-RECQL4) and RECQL5 (AAV-298-RECQL5) (sequences provided above in Table 3). GFP editing frequencies were measured by flow cytometry to identify GFP positive cells and normalized to cells treated with a scrambled shRNA control.

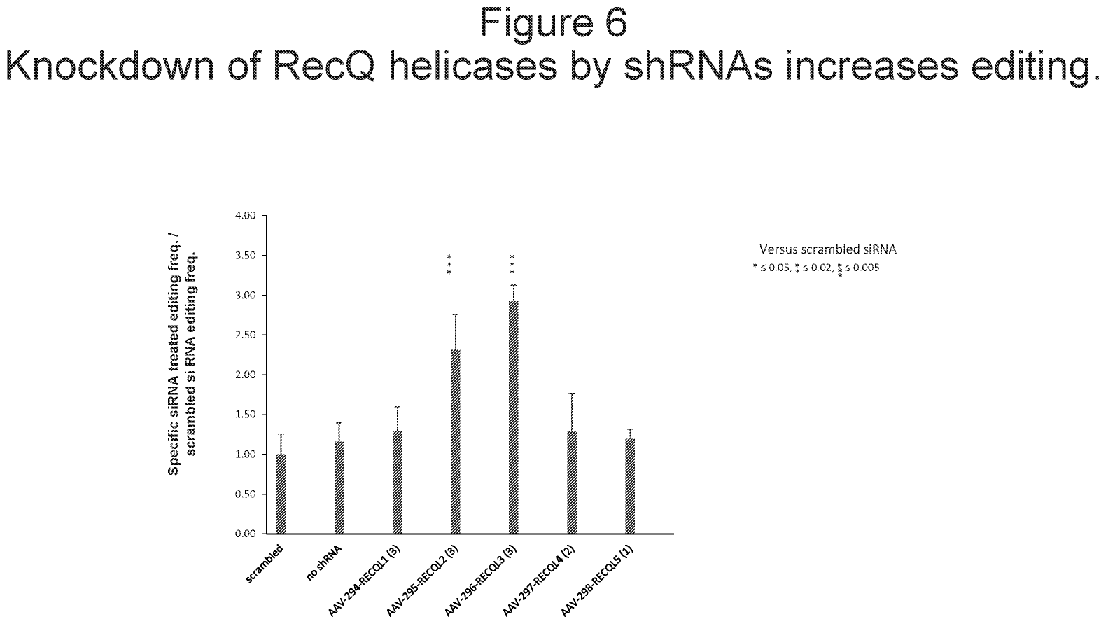

As demonstrated in FIG. 6, inhibition of RecQ helicases by siRNAs increases gene editing.

### Example 8

Inhibition of Mismatch Repair (MMR) Protein by siRNAs Increases Editing

To determine the effect of siRNAs corresponding to the MMR genes expressing MMR proteins PMS2, PMS2, MLH1, MLH2, MLH3, MSH4, MSH5 and MSH6 on gene editing, experiments were performed using the HT-1080 neo/HPRT editing line. Each MMR gene was treated with three different siRNA pairs (provided at Table 4 below).

TABLE 4

| siRNA | Sequence (5′ to 3′) |
|---|---|
| PMS1-si167 | GCTGACATCGTTCTTAGTA (SEQ ID NO: 43) |
| PMS1-si168 | TACTAAGAACGATGTCAGC (SEQ ID NO: 44) |
| PMS1-si169 | GCAGGAAGCTGCTCTGTLA (SEQ ID NO: 45) |
| PMS1-si170 | TAACAGAGCAGCTTCCTGC (SEQ ID NO: 46) |
| PMS1-si171 | GGAATCATCTGGAAAGAAT (SEQ ID NO: 47) |
| PMS1-si172 | ATTCTTTCCAGATGATTCC (SEQ ID NO: 48) |
| PMS2-Si102 | CCGTAGTCACTGTATGGAAT (SEQ ID NO: 49) |
| PMS2-si103 | ATTCCATACAGTGACTACGG (SEQ ID NO: 50) |
| PMS2-si105 | GGGCTAAACTGATTTCCTT (SEQ ID NO: 51) |
| PMS2-si106 | AAGGAAATCAGTTTAGCCC (SEQ ID NO: 52) |
| PMS2-Si161 | GTCTTACATGCATACTGTA (SEQ ID NO: 53) |
| PMS2-Si162 | TACAGTATGCATGTAAGAC (SEQ ID NO: 54) |
| MLH1-si131 | GCCAGCTAATGCTATCAAA (SEQ ID NO: 55) |
| MLH1-si132 | TTTGATAGCATTAGCTGGC (SEQ ID NO: 56) |
| MLH1-si133 | GCATTAGTTTCTCACTTAA (SEQ ID NO: 57) |
| MLH1-si134 | TTAACTGAGAAACTAATGC (SEQ ID NO: 58) |
| MLH1-si135 | GTATTCAAGTGATTGTTAA (SEQ ID NO: 59) |
| MLH1-si136 | TTAACAATCACTTGAATAC (SEQ ID NO: 60) |
| MLH3-si173 | GGATGTTGTACTTGAGAAT (SEQ ID NO: 61) |
| MLH3-si174 | ATTCTCAAGTACAACATCC (SEQ ID NO: 62) |
| MLH3-si175 | GCACAGAAGGTTGCTATAT (SEQ ID NO: 63) |
| MLH3-si176 | ATATAGCAACCTTCTGTGC (SEQ ID NO: 64) |
| MLH3-si177 | GTCTTCAAGTTGAACCTGA (SEQ ID NO: 65) |
| MLH3-si178 | TCAGGTTCAACTTGAAGAC (SEQ ID NO: 66) |
| MSH2-si137 | CCCAGGATGCCATTGTTAA (SEQ ID NO: 67) |
| MSH2-si138 | TTAACAATGGCATCCTGGG (SEQ ID NO: 68) |
| MSH2-si139 | GACTTTACAAGAAGATTTA (SEQ ID NO: 69) |
| MSH2-si140 | TAAATCTTCTTGTAAAGTC (SEQ ID NO: 70) |
| MSH2-si141 | GAAATCATTTCACGAATAA (SEQ ID NO: 71) |
| MSH2-si142 | TTATTCGTGAAATGATTTC (SEQ ID NO: 72) |
| MSH3-si149 | CTCTTTGGCTGCCATCATA (SEQ ID NO: 73) |
| MSH3-si150 | TATGATGGCAGCCAAAGAG (SEQ ID NO: 74) |
| MSH3-si151 | GACTCTGTAGCATTTATCA (SEQ ID NO: 75) |
| MSH3-si152 | TGATAAATGCTACAGAGTC (SEQ ID NO: 76) |
| MSH3-si153 | GCCAGTTTGTGAACTAGAA (SEQ ID NO: 77) |
| MSH3-si154 | TTCTAGTTCACAAACTGGC (SEQ ID NO: 78) |
| MSH4-si179 | GCCTCTAGTTGATATTGAA (SEQ ID NO: 79) |
| MSH4-si180 | TTCAATATCAACTAGAGGC (SEQ ID NO: 80) |
| MSH4-si181 | GCATTTACACTGTTTGCTA (SEQ ID NO: 81) |
| MSH4-si182 | TAGCAAACAGTGTAAATGC (SEQ ID NO: 82) |
| MSH4-si183 | GCTGCTGAATCAAAGATAA (SEQ ID NO: 83) |
| MSH4-si184 | TTATCTTTGATTCAGCAGC (SEQ ID NO: 84) |
| MSH5-si185 | GCCAGTGACTTTGAGATTA (SEQ ID NO: 85) |
| MSH5-si186 | TAATCTCAAAGTCACTGGC (SEQ ID NO: 86) |
| MSH5-si187 | GCTGGTCCTTATTGATGAA (SEQ ID NO: 87) |
| MSH5-si188 | TTCATCAATAAGGACCAGC (SEQ ID NO: 88) |
| MSH5-si189 | GCCCTCTTTGTTTCCTTAT (SEQ ID NO: 89) |
| MSH5-si190 | ATAAGGAAACAAAGAGGGC (SEQ ID NO: 90) |
| MSH6-si143 | CAGATGAAGCCTTAAATAA (SEQ ID NO: 91) |
| MSH6-si144 | TTATTTAAGGCTTCATCTG (SEQ ID NO: 92) |
| MSH6-si145 | GCTCTGATGTGGAATTTAA (SEQ ID NO: 93) |
| MSH6-si146 | TTAAATTCCACATCAGAGC (SEQ ID NO: 94) |
| MSH6-si147 | CTATTACGTTCCTCTATAA (SEQ ID NO: 95) |
| MSH6-si148 | TTATAGAGGAACGTAATAG (SEQ ID NO: 96) |
| Scrambled- | ATTGATCCGATTCGATTGC (SEQ ID NO: 97) |
| si241 | |
| Scrambled- | GCAATCGAATCGGATCAAT (SEQ ID NO: 98) |
| si242 | |

HPRT and neo gene editing frequencies were measured by G418 or HAT selection and normalized to a scrambled siRNA control.

10⁵ HT-1080 cells were plated in 6-well plate on day 1. All experiments were carried out in triplicate. On day 2, cells were transduced with AAV-HSN or AAV-HPe3 at an MOI of 10⁴. 3 nM siRNA mixed with lipofectamine RNAiMAX reagent were then added to the cells. On day 4, the cells were treated with trypsin and 0.1% of the cells were replated without selection in 6-well plates to determine the number of colony forming units. The remainder of the cells were replated in 6-well plates for further analysis of gene editing frequency.

On day 5, neo gene correction experiments were performed by adding G418 (700 ug/ml) to the experimental plates. On day 15, plates were washed with PBS and the colonies were stained with Coomassie brilliant blue G. The percentage of neo gene correction was calculated as the number of G418-resistant CFU per total CFU for each original well.

On day 5, HPRT gene correction experiments were performed by adding 1× HAT (hypoxanthine-aminopterin-thymidine medium) to the experimental plates. On day 15, plates were washed with PBS and the colonies were stained with Coomassie brilliant blue G. The percentage of HP RT gene correction was calculated as the number of HAT-resistant CFU per total CFU for each original well.

The CFU were normalized to the CFU of the control (scrambled siRNA) sample. The editing index was calculated by multiplying the normalized CFU by the mean of the percentage of neo gene correction.

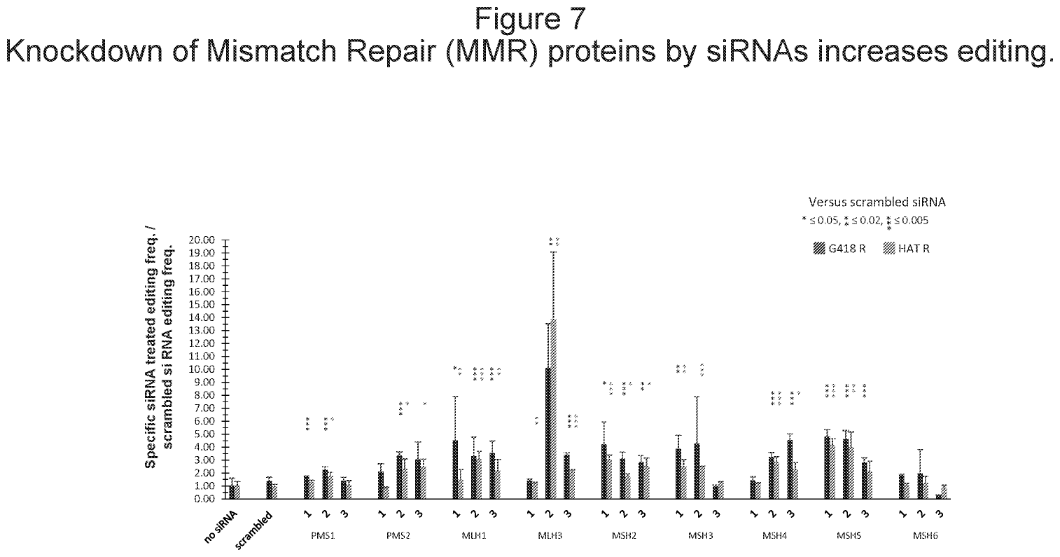

As demonstrated in FIG. 7, inhibition of mismatch repair proteins by siRNAs increases gene editing.

### Example 9

Inhibition of Mismatch Repair (MMR) Protein Combinations by siRNAs Increases Editing

To determine the effect of siRNAs corresponding to the MMR genes expressing MMR proteins PMS2, PMS2, MLH1, MLH2, MLH3, MSH4, MSH5 and MSH6 on gene editing, experiments were performed using the HT-1080 neo/HPRT editing line. Each MMR gene or combination of genes (MSH2 and MSH3, MSH4 and MSH5, MSH2 and MSH6, MLH1 and PMS2, MLH1 and MLH3 and MLH1 and PMS1) was treated with three different siRNA pairs having the sequences presented in Table 5.

TABLE 5

| siRNA | Sequence (5′ to 3′) |
|---|---|
| PMS1-si169 | GCAGGAAGCTGCTCTGTTA (SEQ ID NO: 45) |
| PMS1-si170 | TAACAGAGCAGCTTCCTGC (SEQ ID NO: 46) |
| PMS2-si105 | GGGCTAAACTGATTTCCTT (SEQ ID NO: 51) |
| PMS2-si106 | AAGGAAATCAGTTTAGCCC (SEQ ID NO: 52) |
| MLH1-si133 | GCATTAGTTTCTCAGTTAA (SEQ ID NO: 57) |
| MLH1-si134 | TTAACTGAGAAACTAATGC (SEQ ID NO: 58) |
| MLH3-si175 | GCACAGAAGGTTGCTATAT (SEQ ID NO: 63) |
| MLH3-si176 | ATATAGCAACCTTCTGTGC (SEQ ID NO: 64) |
| MSH2-si137 | CCCAGGATGCCATTGTTAA (SEQ ID NO: 67) |
| MSH2-si138 | TTAACAATGGCATCCTGGG (SEQ ID NO: 68) |
| MSH3-si149 | CTCTTTGGCTGCCATCATA (SEQ ID NO: 73) |
| MSH3-si150 | TATGATGGCAGCCAAAGAG (SEQ ID NO: 74) |
| MSH4-siI83 | GCTGCTGAATCAAAGATAA (SEQ ID NO: 83) |
| MSH4-si184 | TTATCTTTGATTCAGCAGC (SEQ ID NO: 84) |
| MSH5-si185 | GCCAGTGACTTTGAGATTA (SEQ ID NO: 85) |
| MSH5-si186 | TAATCTCAAAGTCACTGGC (SEQ ID NO: 86) |
| MSH6-si143 | CAGATGAAGCCTTAAATAA (SEQ ID NO: 91) |
| MSH6-siI44 | TTATTTAAGGCTCATCTG (SEQ ID NO: 92) |
| Scrambled- | ATTGATCCGATTCGATTGC (SEQ ID NO: 97) |
| si241 | |
| Scrambled- | GCAATCGAATCGGATCAAT (SEQ ID NO: 98) |
| si242 | |

HPRT and neo gene editing frequencies were measured by G418 or HAT selection and normalized to a scrambled siRNA control as follows.

10⁵ HT-1080 cells were plated in 6-well plates on day 1. All experiments were carried out in triplicate. On day 2, cells were transduced with AAV-HSN or AAV-HPe3 at an MOI of 10⁴. 3 nM siRNA mixed with lipofectamine RNAiMAX reagent were then added to the cells. On day 4, the cells were treated with trypsin and 0.1% of the cells were replated without selection in 6-well plates to determine the number of colony forming units. The remainder of the cells were replated in 6-well plates for further analysis of gene editing frequency.

On day 5, neo gene correction experiments were performed by adding G418 (700 ug/ml) to the experimental plates. On day 15, plates were washed with PBS and the colonies were stained with Coomassie brilliant blue G. The percentage of neo gene correction was calculated as the number of G418-resistant CFU per total CFU for each original well.

On day 5, HPRT gene correction experiments were performed by adding 1× HAT is (hypoxanthine-aminopterin-thymidine medium) to the experimental plates. On day 15, plates were washed with PBS and the colonies were stained with Coomassie brilliant blue G. The percentage of HPRT gene correction was calculated as the number of HAT-resistant CFU per total CFU for each original well. The CFU were normalized to the CFU of the control (scrambled siRNA) sample. The editing index was calculated by multiplying the normalized CFU by the mean of the percentage of neo gene correction.

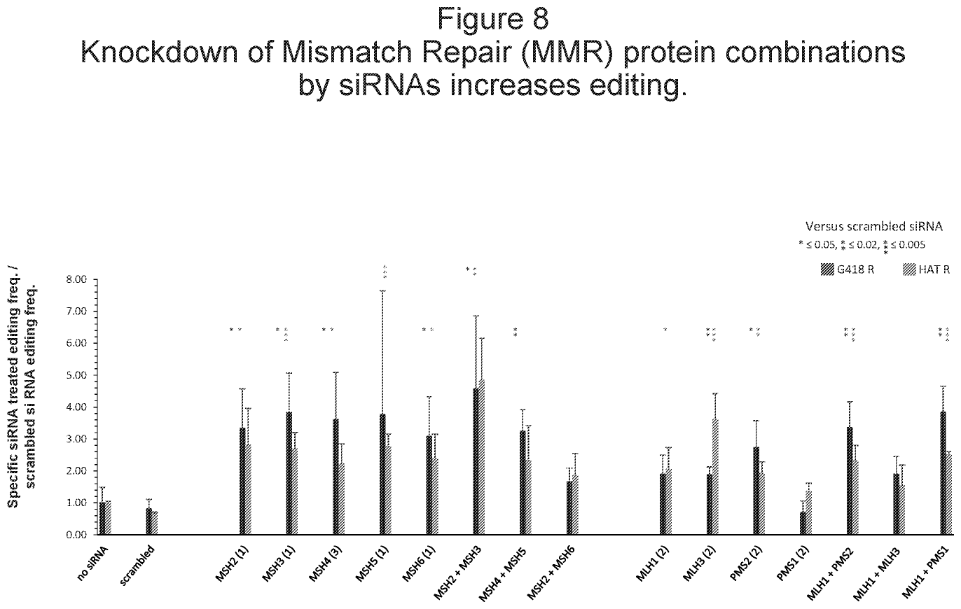

As demonstrated in FIG. 8, inhibition of mismatch repair protein combinations by siRNAs increases gene editing.

### Example 10

Inhibition of PCNA by shRNAs Increases Editing

To determine the effect of shRNA specific for the mRNA corresponding to the PCNA gene, on gene editing, experiments were performed using the HT-1080 GFP editing line. The PCNA gene was treated with a lentivirus vector expressing a shRNA specific for the PCNA gene (AAV-301-PCNA) (sequences provided in Table 6 below). A map of the shRNA vector is provided in FIG. 10. HPRT and neo editing frequencies were measured by G418 or HAT selection, and normalized to a scrambled shRNA control.

TABLE 6

| shRNA | |
|---|---|
| PCNA-320 | TTGTAATTTCCTGTGCAATTGGATCCAATTGCACAGGAAAT |
| | TACAACTTTTTC (SEQ ID NO: 99) |
| PCNA-321 | TCGAGAAAAAGTTGTAATTTCCTGTGCAATMGATCCAATTG |
| | CACAGGAAATTACAA (SEQ ID NO: 100) |

GFP editing frequencies were measured by flow cytometry to identify GFP positive cells and normalized to cells treated with a scrambled siRNA control.

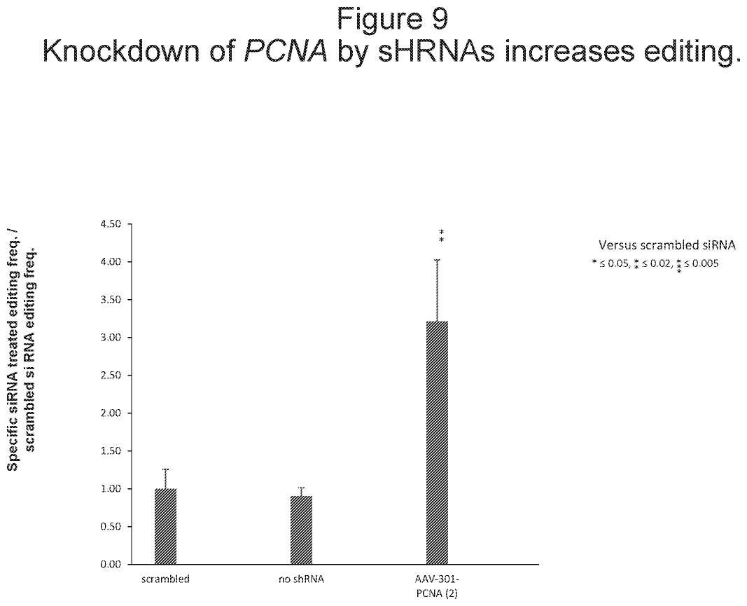

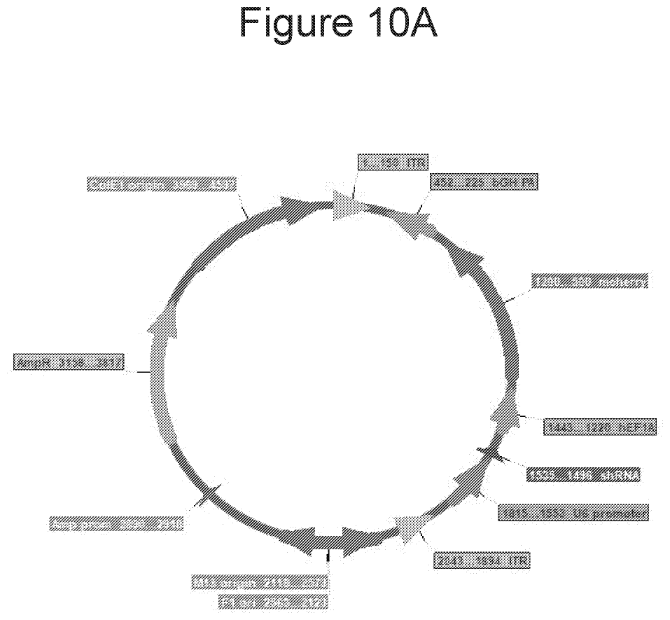

As demonstrated in FIG. 9, inhibition of PCNA by shRNAs increases gene editing.

### Example 11

Inhibition of Additional Selected Fork Proteins by shRNAs Increases Editing

To determine the effect of shRNA specific for selected replication fork proteins on gene editing, experiments were performed using the HT-1080/neo/HPRT editing lines. Each gene indicated in Table 8 below was inhibited with a shRNA (sequences provided in Table 7 below) expressed from a lentivirus vector.

TABLE 7

| shRNA cloned | |
|---|---|
| in pTRIPZ | |
| (Dharmacon) | Sequence (5′ to 3′) |
| POLE4-SH7 | GTTGACAGTGAGCGCGGAGAGACTTGGATAATGCAATAGTGAAGC |
| | CACAGATGTATTGCATTATCCAAGTCTCTCCTTGCCTACTGCCTC |
| | (SEQ ID NO: 101) |
| ANAPC5-SH9 | GTTGACAGTGAGCGCGCGCATTATCTCAGCTACTTATAGTGAAGC |
| | CACAGATGTATAAGTAGCTGAGATAATGCGCTTGCCTACTGCCT |
| | C (SEQ ID NO: 102) |
| PMS2-SHI5 | GTTGACAGTGAGCGCCCGTAGTCACTGTATGGAATATAGTGAAGC |
| | CACAGATGTATATTCCATACAGTGACTACGGTTGCCTACTGCCT |
| | C (SEQ ID NO: 103) |
| BLM-SH39 | GTTGACAGTGAGCGTAAGCAGCGATGTGATTTGCATTAGTGAAGC |
| | CACAGATGTAATGCAAATCACATCGCTGCTTA |
| | TGCCTACTGCCTC (SEQ ID NO: 104) |
| RMII/BLAP75- | GTTGACAGTGAGCGTGATCTAGTTACAGCTGAAGCATAGTGAAGC |
| SH40 | CACAGATGTATGCTTCAGCTGTAACTAGATCA |
| | TGCCTACTGCCTC (SEQ ID NO: 105) |
| Topo III | GTTGACAGTGAGCGTGGACATTTACTGGCTCATGATTAGTGAAGC |
| alpha-SH41 | CACAGATGTAATCATGAGCCAGTAAATGTCCA |
| | TGCCTACTGCCTC (SEQ ID NO: 106) |
| MEIS2-SH42 | GTTGACAGTGAGCGCGGACTTTATCAGAAACTCAAATAGTGAAGC |
| | CACAGATGTATTTGAGTTTCTGATAAAGTCCTTGCCTACTGCCTC |
| | (SEQ ID NO: 107) |
| ORC1L-SH56 | GTTGACAGTGAGCGCGCCACGTTTCAACAGATATATTAGTGAAGC |
| | CACAGATGTAATATATCTGTTGAAACGTGGCTTGCCTACTGCCT |
| | C (SEQ ID NO: 108) |
| BLM-SH60 | GTTGACAGTGAGCGCGGAGTCTGCGTGCGAGGATTATAGTGAAGC |
| | CACAGATGTATAATCCTCGCACGCAGACTCCTTGCCTACTGCCT |
| | C (SEQ ID NO: 109) |
| BLM-SH61 | GTTGACAGTGAGCGAAACCTTCCTATGATATTGATATAGTGAAGCC |
| | ACAGATGTATATCAATATCATAGGAAGGTTGTGCCTACTGCCTC |
| | (SEQ ID NO: 110) |
| BLM-SH62 | GTTGACAGTGAGCGCGGTGTCCATTACTTCAATATTTAGTGAAGCC |
| | ACAGATGTAAATATTGAAGTAATGGACACCATGCCTACTGCCTC |
| | (SEQ ID NO: 111) |
| BLM-SH63 | GTTGACAGTGAGCGAAGGTTATCTGTGCTACAATTGTAGTGAAGCC |
| | ACAGATGTACAATTGTAGCACAGATAACCTGTGCCTACTGCCTC |
| | (SEQ ID NO: 112) |
| POLA1-SH79 | GTTGACAGTGAGCGCGCAGATCATGTCTTGAGCTAAATAGTGAAGC |
| | CACAGATGTATTTAGCTCACACATGATCTGCATGCCTACTGCCTC |
| | (SEQ ID NO: 113) |

HPRT and neo gene editing frequencies were measured by G418 or HAT selection and normalized to a cell that was not treated with a shRNA.

Cells were first transduced with a lentivirus expressing a shRNA specific for the targeted genes. 2×10⁴ HT-1080 cells transduced with the specific shRNAs were then plated in 6-well plates on day 1, and the expression of the shRNA was induced for 24 hours with doxycycline (1 μg/ml). On day 2, cells were transduced with AAV-HSN or AAV-HPe3 at a MOI of 2×10⁴. On day 4, the cells were treated with trypsin and 0.1% of the cells were replated without selection in 6-well plates to determine the number of colony forming units. The remainder of the cells were replated in 6-well plates for further analysis of gene editing frequency.

On day 5, neo gene correction experiments were performed by the addition of G418 (700 ug/ml) to the experimental plates. On day 16, plates were washed with PBS and the colonies were stained with Coomassie brilliant blue G. The percentage of neo gene correction was calculated as the number of G418-resistant CFU per total CFU for each original well. On day 6, HPRT gene correction experiments were performed by adding 1× HAT (hypoxanthine-aminopterin-thymidine medium) to the experimental plates. On day 16, plates were washed with PBS and the colonies were stained with Coomassie brilliant blue G. The percentage of HPRT gene correction was calculated as the number of HAT-resistant CFU per total CFU for each original well.

The results of the experiments are provided in Table 8 below.

TABLE 8

| shRNA | Target Gene | G418 (neo) | HAT (HPRT) |
|---|---|---|---|
| None | None | 1.00 | 1.00 |
| SH7 | 1B3, POLE4 | 1.57 | 8.24 |
| SH9 | 1B5, anapc5 | 2.81 | 29.10 |
| sh15 | 2b3, pms2 | 3.88 | 17.59 |
| Sh39 | BLM | 1.67 | 3.8 |
| Sh40 | RMI1/BLAP75 | 1.37 | 1.61 |
| Sh41 | Topo III alpha | 1.42 | 1.66 |
| Sh42 | 12A5, MEIS2 | 1.26 | ND |
| SH56 | 12B12, ORC1L | 2.07 | 2.11 |
| SH60 | BLM | 2.08 | 1.38 |
| SH61 | BLM | 2.64 | 2.82 |
| SH62 | BLM | 2.28 | 2.34 |
| SH63 | BLM | 1.99 | 0.92 |
| SH79 | POLA1 | 1.24 | 2.46 |

What is claimed is:

1. A method of editing a gene in a cell comprising modulating replication fork function in the cell, and editing the gene in the cell.

2. The method of claim 1, further comprising contacting the cell with a replication fork modulator.

3. The method of claim 1 or 2, further comprising contacting the cell with a gene editing vector.

4. The method of claim 3, wherein the gene editing vector is an adeno-associated virus (AAV) vector.

5. The method of any one of claims 2-4, wherein the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

6. The method of any one of claims 2-5, wherein the replication fork modulator is emetine.

7. The method of any one of claims 2-6, wherein the replication fork modulator is siRNA.

8. The method of any one of claims 2-7, wherein the replication fork modulator is shRNA.

9. The method of any one of claims 1-8, wherein the replication fork function is DNA synthesis.

10. The method of any one of claims 2-9, wherein the replication fork modulator is a leading strand synthesis inhibitor.

11. The method of any one of claims 2-10, wherein the replication fork modulator is a lagging strand synthesis inhibitor.

12. The method of any one of claims 1-11, further comprising wherein modulating replication fork function comprises modulating the function or level of expression of a replication fork protein.

13. The method of claim 12, wherein the replication fork protein is selected from the group consisting of: DNA polymerase α, DNA primase, RNA primase, DNA polymerase ε, DNA polymerase δ, fork protection complex (FPC) components Timeless, Tipin, Claspin and And1, Cdc45, MCM 2-7 (mini-chromosome maintenance) helicase 2-7 hexamer proteins (Mcm2, Mcm3, Mcm4, Mcm5, Mcm6 and Mcm7), go-ichi-ni-san (GINS) complex proteins (Sld5, Psf1, Psf2 and Psf3), replication protein A (RPA), replication factor C clamp loader (RFC) proteins (Rfc1, Rfc2, Rfc3, Rfc4, and Rfc5), RM1I protein, ATR kinase, ATR-interacting protein (ATRIP), RecQ Helicase proteins (RECQL1, RECQL2, RECQL3, RECQL4 and RECQL5), Mismatch Repair (MMR) proteins (PMS2, PMS2, MLH1, MLH2, MLH3, MSH4, MSH5 and MSH6), Proliferating cell nuclear antigen (PCNA), Flap endonuclease 1 (FEN1), DNA ligase, anaphase promoting complex subunit 5, RecQ-mediated genome instability protein 1, Origin recognition complex subunit 1, Homeobox protein Meis2, DNA Topoisomerase III Alpha, DNA polymerase epsilon 4, and FANCM protein.

14. The method of claim 13, wherein the replication fork protein is selected from the group consisting of: RecQ Helicase proteins (RECQL1, RECQL2, RECQL3, RECQL4 and RECQL5), Mismatch Repair (MMR) proteins (PMS2, PMS2, MLH1, MLH2, MLH3, MSH4, MSH5 and MSH6), and Proliferating cell nuclear antigen (PCNA).

15. The method of claim 13 or 14, wherein the replication fork protein is a RecQ Helicase protein selected from the group consisting of: RECQL1, RECQL2, RECQL3, RECQL4 and RECQL5.

16. The method of claim 13 or 14, wherein the replication fork protein is a Mismatch Repair (MMR) protein selected from the group consisting of: PMS2, PMS2, MLH1, MLH2, MLH3, MSH4, MSH5 and MSH6.

17. The method of claim 13 or 14, wherein the replication fork protein is Proliferating cell nuclear antigen (PCNA).

18. The method of any one of claims 13-15, wherein the replication fork protein is a RecQ Helicase protein selected from the group consisting of: RECQL1, RECQL2, RECQL3, RECQL4 and RECQL5, and the replication fork modulator is emetine.

19. The method of any one of claims 13-15, wherein the replication fork protein is a RecQ Helicase protein selected from the group consisting of: RECQL1, RECQL2, RECQL3, RECQL4 and RECQL5, and the replication fork modulator is siRNA.

20. The method of any one of claims 13-15, wherein the replication fork protein is a RecQ Helicase protein selected from the group consisting of: RECQL1, RECQL2, RECQL3, RECQL4 and RECQL5, and the replication fork modulator is shRNA.

21. The method of claim 13 or 16, wherein the replication fork protein is a Mismatch Repair (MMR) protein selected from the group consisting of: PMS2, PMS2, MLH1, MLH2, MLH3, MSH4, MSH5 and MSH6, and the replication fork modulator is emetine.

22. The method of claim 13 or 16, wherein the replication fork protein is a Mismatch Repair (MMR) protein selected from the group consisting of: PMS2, PMS2, MLH1, MLH2, MLH3, MSH4, MSH5 and MSH6, and the replication fork modulator is siRNA.

23. The method of claim 13 or 16, wherein the replication fork protein is a Mismatch Repair (MMR) protein selected from the group consisting of: PMS2, PMS2, MLH1, MLH2, MLH3, MSH4, MSH5 and MSH6, and the replication fork modulator is shRNA.

24. The method of claim 13 or 17, wherein the replication fork protein is Proliferating cell nuclear antigen (PCNA) and the replication fork modulator is emetine.

25. The method of claim 13 or 17, wherein the replication fork protein is Proliferating cell nuclear antigen (PCNA) and the replication fork modulator is siRNA

26. The method of claim 13 or 17, wherein the replication fork protein is Proliferating cell nuclear antigen (PCNA) and the replication fork modulator is shRNA.

27. The method of any one of claims 1-26, wherein the cell is selected from the group consisting of: pluripotent stem cell, induced pluripotent stem cell, and embryonic stem cell.

28. The method of any one of claims 1-27, wherein the cell is a primate cell.

29. The method of any one of claims 1-26 and 28, wherein the cell is a differentiated cell.

30. The method of any one of claims 2-29, wherein the gene editing efficiency in the cell is greater than the gene editing efficiency in a cell that has not been contacted with a replication fork modulator.

31. A method of editing a gene in a cell comprising contacting a cell with a replication fork modulator for a period of time before editing the gene in the cell, and editing the gene in the cell.

32. The method of claim 31, wherein the period of time is 8 hours to 7 days.

33. A method of editing a gene in a cell comprising contacting a cell with a replication fork modulator during gene editing, and editing the gene in the cell.

34. A method of editing a gene in a cell comprising contacting a cell with a replication fork modulator for a period of time after editing the gene in the cell.

35. The method of any one of claims 31-34, wherein the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

36. The method of any one of claims 31-35, wherein the replication fork modulator is a leading strand synthesis inhibitor.

37. The method of any one of claim 31-36, wherein the replication fork modulator is a lagging strand synthesis inhibitor.

38. The method of any one of claims 31-37, further comprising contacting the cell with a gene editing vector.

39. The method of claim 38, wherein the gene editing vector is an adeno-associated virus (AAV) vector.

40. A method of editing a gene in a cell of a subject, comprising:
a. administering a gene editing vector to the subject; and
b. administering a replication fork modulator to the subject.

41. The method of claim 40, wherein the replication fork modulator is administered after the gene editing vector is administered.

42. The method of claim 40, wherein the replication fork modulator is administered before the gene editing vector is administered.

43. The method of claim 40, wherein the gene editing vector and the replication fork modulator are administered at the same time.

44. The method of any one of claims 40-43, wherein the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

45. The method of any one of claims 40-44, wherein the replication fork modulator is a leading strand synthesis inhibitor.

46. The method of any one of claims 40-45, wherein the replication fork modulator is a lagging strand synthesis inhibitor.

47. The method of any one of claims 40-46, wherein the gene editing vector is an adeno-associated virus (AAV) vector.

48. A method of editing a gene in a cell comprising:
a. editing the gene in the cell; and
b. contacting the gene edited cell with a replication fork modulator for a period of time.

49. The method of claim 48, wherein the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

50. The method of claim 48 or 49, wherein the replication fork modulator is a leading strand synthesis inhibitor.

51. The method of claims 48-50, wherein the replication fork modulator is a lagging strand synthesis inhibitor.

52. The method of any one of claims 48-51, further comprising contacting the cell with a gene editing vector.

53. The method of claim 52, wherein the gene editing vector is an adeno-associated virus (AAV) vector.

54. The method of any one of claims 31-53, wherein the replication fork protein is selected from the group consisting of: RecQ Helicase proteins (RECQL1, RECQL2, RECQL3, RECQL4 and RECQL5), Mismatch Repair (MMR) proteins (PMS2, PMS2, MLH1, MLH2, MLH3, MSH4, MSH5 and MSH6), and Proliferating cell nuclear antigen (PCNA).

55. The method of any one of claims 31-54, wherein the replication fork protein is a RecQ Helicase protein selected from the group consisting of: RECQL1, RECQL2, RECQL3, RECQL4 and RECQL5.

56. The method of any one of claims 31-53, wherein the replication fork protein is a Mismatch Repair (MMR) protein selected from the group consisting of: PMS2, PMS2, MLH1, MLH2, MLH3, MSH4, MSH5 and MSH6.

57. The method of any one of claims 31-53, wherein the replication fork protein is Proliferating cell nuclear antigen (PCNA).

58. The method of any one of claims 31-54, wherein the replication fork protein is a RecQ Helicase protein selected from the group consisting of: RECQL1, RECQL2, RECQL3, RECQL4 and RECQL5, and the replication fork modulator is emetine.

59. The method of any one of claims 31-54, wherein the replication fork protein is a RecQ Helicase protein selected from the group consisting of: RECQL1, RECQL2, RECQL3, RECQL4 and RECQL5, and the replication fork modulator is siRNA.

60. The method of any one of claims 31-54, wherein the replication fork protein is a RecQ Helicase protein selected from the group consisting of: RECQL1, RECQL2, RECQL3, RECQL4 and RECQL5, and the replication fork modulator is shRNA.

61. The method of any one of claims 31-53 and 56, wherein the replication fork protein is a Mismatch Repair (MMR) protein selected from the group consisting of: PMS2, PMS2, MLH1, MLH2, MLH3, MSH4, MSH5 and MSH6, and the replication fork modulator is emetine.

62. The method of any one of claims 31-53 and 56, wherein the replication fork protein is a Mismatch Repair (MMR) protein selected from the group consisting of: PMS2, PMS2, MLH1, MLH2, MLH3, MSH4, MSH5 and MSH6, and the replication fork modulator is siRNA.

63. The method of any one of claims 31-53 and 56, wherein the replication fork protein is a Mismatch Repair (MMR) protein selected from the group consisting of PMS2, PMS2, MLH1, MLH2, MLH3, MSH4, MSH5 and MSH6, and the replication fork modulator is shRNA.

64. The method of any one of claims 31-53 and 57, wherein the replication fork protein is Proliferating cell nuclear antigen (PCNA) and the replication fork modulator is emetine.

65. The method of any one of claims 31-53 and 57, wherein the replication fork protein is Proliferating cell nuclear antigen (PCNA) and the replication fork modulator is siRNA

66. The method of any one of claims 31-53 and 57, wherein the replication fork protein is Proliferating cell nuclear antigen (PCNA) and the replication fork modulator is shRNA.

67. The method of any one of claims 31-66, wherein the cell is selected from the group consisting of: pluripotent stem cell, induced pluripotent stem cell, and embryonic stem cell.

68. The method of any one of claims 31-66, wherein the cell is a primate cell.

69. The method of any one of claims 31-66 and 68 wherein the cell is a differentiated cell.

70. A composition comprising a population of gene edited cells obtained by modulating replication fork function in the cells.

71. The composition of claim 70, wherein the gene editing efficiency in the population of cells is greater than in a second population of cells gene edited in the absence of modulating replication fork function in the cells.

72. The composition of claim 70 or 71, wherein the population of gene edited cells are obtained by modulating replication fork function in the cells by contacting the cells with a replication fork modulator.

73. The composition of claim 72, wherein the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

74. The composition of claim 72 or 73, wherein the replication fork modulator is a leading strand synthesis inhibitor.

75. The composition of any one of claims 72-73, wherein replication fork modulator is a lagging strand synthesis inhibitor.

76. A cell comprising a gene editing vector and an exogenous replication fork modulator.

77. The cell of claim 76, wherein the gene editing vector is an adeno-associated virus (AAV) vector.

78. A cell comprising a gene modification and an exogenous replication fork modulator.

79. The cell of any one of claims 76-78, wherein the replication fork modulator is selected from the group consisting of emetine, dehydroemetine, emetine dihydrochloro hydrate, cephaeline, or salts thereof; an shRNA, siRNA, aptamer, small internally segmented interfering RNA, microRNA, antisense oligonucleotide, or antibody specific for RecQ helicase; an shRNA, siRNA, aptamer, small internally segmented interfering RNA, microRNA, antisense oligonucleotide, or antibody specific for PCNA; and an shRNA, siRNA, aptamer, small internally segmented interfering RNA, microRNA, antisense oligonucleotide, or antibody specific for a mismatch repair protein.

80. The cell of any one of claims 76-79, wherein the replication fork modulator is a leading strand synthesis activator or a leading strand synthesis inhibitor.

81. The cell of any one of claims 76-80, wherein the replication fork modulator is a lagging strand synthesis activator or a lagging strand synthesis inhibitor.

82. A cell that is derived or differentiated from the cell of any one of claims 76-81.

83. A method of treating a disease in a subject in need comprising administering to the subject an effective amount of the cell of any one of claims 70-82.

84. A method of transplantation in a subject in need comprising administering to the subject an effective amount of the cell of any one of claims 70-82.


SEQUENCE LISTING (113 sequences)

SEQ ID NO: 1 - DNA, 19 bp, Artificial Sequence
  Synthetic oligonucleotide
ctcactgagtgatacttta

SEQ ID NO: 2 - DNA, 19 bp, Artificial Sequence
  Synthetic oligonucleotide
taaagtatcactcagtgag

SEQ ID NO: 3 - DNA, 19 bp, Artificial Sequence
  Synthetic oligonucleotide
caatctggttctaagaata

SEQ ID NO: 4 - DNA, 19 bp, Artificial Sequence
  Synthetic oligonucleotide
tattcttagaaccagattg

SEQ ID NO: 5 - DNA, 19 bp, Artificial Sequence
  Synthetic oligonucleotide
gtcagtgttgtattggaaa

SEQ ID NO: 6 - DNA, 19 bp, Artificial Sequence
  Synthetic oligonucleotide
tttccaatacaacactgac

SEQ ID NO: 7 - DNA, 18 bp, Artificial Sequence
  Synthetic oligonucleotide
agcagcgatgtgatttgc

SEQ ID NO: 8 - DNA, 18 bp, Artificial Sequence
  Synthetic oligonucleotide
gcaaatcacatcgctgct

SEQ ID NO: 9 - DNA, 19 bp, Artificial Sequence
  Synthetic oligonucleotide
accttcctatgatattgat

SEQ ID NO: 10 - DNA, 19 bp, Artificial Sequence
  Synthetic oligonucleotide
atcaatatcataggaaggt

SEQ ID NO: 11 - DNA, 19 bp, Artificial Sequence
  Synthetic oligonucleotide
gtgtccattacttcaatat

SEQ ID NO: 12 - DNA, 19 bp, Artificial Sequence
  Synthetic oligonucleotide
atattgaagtaatggacac

SEQ ID NO: 13 - DNA, 19 bp, Artificial Sequence
  Synthetic oligonucleotide
ggatgaatgtgcagaataa

SEQ ID NO: 14 - DNA, 19 bp, Artificial Sequence
  Synthetic oligonucleotide
ttattctgcacattcatcc

SEQ ID NO: 15 - DNA, 19 bp, Artificial Sequence
  Synthetic oligonucleotide
ttaacagtctggttaaaca

SEQ ID NO: 16 - DNA, 19 bp, Artificial Sequence
  Synthetic oligonucleotide
tgtttaaccagactgttaa

SEQ ID NO: 17 - DNA, 19 bp, Artificial Sequence
  Synthetic oligonucleotide
gacttcatctgcagagaga

SEQ ID NO: 18 - DNA, 19 bp, Artificial Sequence
  Synthetic oligonucleotide
tctctctgcagatgaagtc

SEQ ID NO: 19 - DNA, 19 bp, Artificial Sequence
  Synthetic oligonucleotide
ggctcaacatgaagcagaa

SEQ ID NO: 20 - DNA, 19 bp, Artificial Sequence
  Synthetic oligonucleotide
ttctgcttcatgttgagcc

SEQ ID NO: 21 - DNA, 19 bp, Artificial Sequence
  Synthetic oligonucleotide
gccactggttccttcacca

SEQ ID NO: 22 - DNA, 19 bp, Artificial Sequence
  Synthetic oligonucleotide
tggtgaaggaaccagtggc

SEQ ID NO: 23 - DNA, 19 bp, Artificial Sequence
  Synthetic oligonucleotide
gggaatctgtcctgcagaa

SEQ ID NO: 24 - DNA, 19 bp, Artificial Sequence
  Synthetic oligonucleotide
ttctgcaggacagattccc

SEQ ID NO: 25 - DNA, 19 bp, Artificial Sequence
  Synthetic oligonucleotide
ggatgaagctcattgtgtt

SEQ ID NO: 26 - DNA, 19 bp, Artificial Sequence
  Synthetic oligonucleotide
aacacaatgagcttcatcc

SEQ ID NO: 27 - DNA, 19 bp, Artificial Sequence
  Synthetic oligonucleotide
gtgttgcgctgcctcttga

SEQ ID NO: 28 - DNA, 19 bp, Artificial Sequence
  Synthetic oligonucleotide
tcaagaggcagcgcaacac

SEQ ID NO: 29 - DNA, 19 bp, Artificial Sequence
  Synthetic oligonucleotide
tctatgatgtgcaattcaa

SEQ ID NO: 30 - DNA, 19 bp, Artificial Sequence
  Synthetic oligonucleotide
ttgaattgcacatcataga

SEQ ID NO: 31 - DNA, 19 bp, Artificial Sequence
  Synthetic oligonucleotide
attgaccgaatcactcgtg

SEQ ID NO: 32 - DNA, 19 bp, Artificial Sequence
  Synthetic oligonucleotide
cacgagtgattcggtcaat

SEQ ID NO: 33 - DNA, 58 bp, Artificial Sequence
  Synthetic oligonucleotide
tcagtgttgtattggaaattggatccaatttccaatacaacactgactttttctcgag

SEQ ID NO: 34 - DNA, 62 bp, Artificial Sequence
  Synthetic oligonucleotide
ggccctcgagaaaaagtcagtgttgtattggaaattggatccaatttccaatacaacactga

SEQ ID NO: 35 - DNA, 47 bp, Artificial Sequence
  Synthetic oligonucleotide
tgtccattacttcaatatttggatccaaatattgaagtaatggacac

SEQ ID NO: 36 - DNA, 62 bp, Artificial Sequence
  Synthetic oligonucleotide
ggccctcgagaaaaagtgtccattacttcaatatttggatccaaatattgaagtaatggaca

SEQ ID NO: 37 - DNA, 58 bp, Artificial Sequence
  Synthetic oligonucleotide
acttcatctgcagagagattggatccaatctctctgcagatgaagtctttttctcgag

SEQ ID NO: 38 - DNA, 62 bp, Artificial Sequence
  Synthetic oligonucleotide
ggccctcgagaaaaagacttcatctgcagagagattggatccaatctctctgcagatgaagt

SEQ ID NO: 39 - DNA, 58 bp, Artificial Sequence
  Synthetic oligonucleotide
ccactggttccttcaccattggatccaatggtgaaggaaccagtggctttttctcgag

SEQ ID NO: 40 - DNA, 62 bp, Artificial Sequence
  Synthetic oligonucleotide
ggccctcgagaaaaagccactggttccttcaccattggatccaatggtgaaggaaccagtgg

SEQ ID NO: 41 - DNA, 58 bp, Artificial Sequence
  Synthetic oligonucleotide
gatgaagctcattgtgttttggatccaaaacacaatgagcttcatcctttttctcgag

SEQ ID NO: 42 - DNA, 63 bp, Artificial Sequence
  Synthetic oligonucleotide
ggccctcgagaaaaaggatgaagctcattgtgttttggatccaaaacacaatgagcttcatcc

SEQ ID NO: 43 - DNA, 19 bp, Artificial Sequence
  Synthetic oligonucleotide
gctgacatcgttcttagta

SEQ ID NO: 44 - DNA, 19 bp, Artificial Sequence
  Synthetic oligonucleotide
tactaagaacgatgtcagc

SEQ ID NO: 45 - DNA, 19 bp, Artificial Sequence
  Synthetic oligonucleotide
gcaggaagctgctctgtta

SEQ ID NO: 46 - DNA, 19 bp, Artificial Sequence
  Synthetic oligonucleotide
taacagagcagcttcctgc

SEQ ID NO: 47 - DNA, 19 bp, Artificial Sequence
  Synthetic oligonucleotide
ggaatcatctggaaagaat

SEQ ID NO: 48 - DNA, 19 bp, Artificial Sequence
  Synthetic oligonucleotide
attctttccagatgattcc

SEQ ID NO: 49 - DNA, 20 bp, Artificial Sequence
  Synthetic oligonucleotide
ccgtagtcactgtatggaat

SEQ ID NO: 50 - DNA, 20 bp, Artificial Sequence
  Synthetic oligonucleotide
attccatacagtgactacgg

SEQ ID NO: 51 - DNA, 19 bp, Artificial Sequence
  Synthetic oligonucleotide
gggctaaactgatttcctt

SEQ ID NO: 52 - DNA, 19 bp, Artificial Sequence
  Synthetic oligonucleotide
aaggaaatcagtttagccc

SEQ ID NO: 53 - DNA, 19 bp, Artificial Sequence
  Synthetic oligonucleotide
gtcttacatgcatactgta

SEQ ID NO: 54 - DNA, 19 bp, Artificial Sequence
  Synthetic oligonucleotide
tacagtatgcatgtaagac

SEQ ID NO: 55 - DNA, 19 bp, Artificial Sequence
  Synthetic oligonucleotide
gccagctaatgctatcaaa

SEQ ID NO: 56 - DNA, 19 bp, Artificial Sequence
  Synthetic oligonucleotide
tttgatagcattagctggc

SEQ ID NO: 57 - DNA, 19 bp, Artificial Sequence
  Synthetic oligonucleotide
gcattagtttctcagttaa

SEQ ID NO: 58 - DNA, 19 bp, Artificial Sequence
  Synthetic oligonucleotide
ttaactgagaaactaatgc

SEQ ID NO: 59 - DNA, 19 bp, Artificial Sequence
  Synthetic oligonucleotide
gtattcaagtgattgttaa

SEQ ID NO: 60 - DNA, 19 bp, Artificial Sequence
  Synthetic oligonucleotide
ttaacaatcacttgaatac

SEQ ID NO: 61 - DNA, 19 bp, Artificial Sequence
  Synthetic oligonucleotide
ggatgttgtacttgagaat

SEQ ID NO: 62 - DNA, 19 bp, Artificial Sequence
  Synthetic oligonucleotide
attctcaagtacaacatcc

SEQ ID NO: 63 - DNA, 19 bp, Artificial Sequence
  Synthetic oligonucleotide
gcacagaaggttgctatat

SEQ ID NO: 64 - DNA, 19 bp, Artificial Sequence
  Synthetic oligonucleotide
atatagcaaccttctgtgc

SEQ ID NO: 65 - DNA, 19 bp, Artificial Sequence
  Synthetic oligonucleotide
gtcttcaagttgaacctga

SEQ ID NO: 66 - DNA, 19 bp, Artificial Sequence
  Synthetic oligonucleotide
tcaggttcaacttgaagac

SEQ ID NO: 67 - DNA, 19 bp, Artificial Sequence
  Synthetic oligonucleotide
cccaggatgccattgttaa

SEQ ID NO: 68 - DNA, 19 bp, Artificial Sequence
  Synthetic oligonucleotide
ttaacaatggcatcctggg

SEQ ID NO: 69 - DNA, 19 bp, Artificial Sequence
  Synthetic oligonucleotide
gactttacaagaagattta

SEQ ID NO: 70 - DNA, 19 bp, Artificial Sequence
  Synthetic oligonucleotide
taaatcttcttgtaaagtc

SEQ ID NO: 71 - DNA, 19 bp, Artificial Sequence
  Synthetic oligonucleotide
gaaatcatttcacgaataa

SEQ ID NO: 72 - DNA, 19 bp, Artificial Sequence
  Synthetic oligonucleotide
ttattcgtgaaatgatttc

SEQ ID NO: 73 - DNA, 19 bp, Artificial Sequence
  Synthetic oligonucleotide
ctctttggctgccatcata

SEQ ID NO: 74 - DNA, 19 bp, Artificial Sequence
  Synthetic oligonucleotide
tatgatggcagccaaagag

SEQ ID NO: 75 - DNA, 19 bp, Artificial Sequence
  Synthetic oligonucleotide
gactctgtagcatttatca

SEQ ID NO: 76 - DNA, 19 bp, Artificial Sequence
  Synthetic oligonucleotide
tgataaatgctacagagtc

SEQ ID NO: 77 - DNA, 19 bp, Artificial Sequence
  Synthetic oligonucleotide
gccagtttgtgaactagaa

SEQ ID NO: 78 - DNA, 19 bp, Artificial Sequence
  Synthetic oligonucleotide
ttctagttcacaaactggc

SEQ ID NO: 79 - DNA, 19 bp, Artificial Sequence
  Synthetic oligonucleotide
gcctctagttgatattgaa

SEQ ID NO: 80 - DNA, 19 bp, Artificial Sequence
  Synthetic oligonucleotide
ttcaatatcaactagaggc

SEQ ID NO: 81 - DNA, 19 bp, Artificial Sequence
  Synthetic oligonucleotide
gcatttacactgtttgcta

SEQ ID NO: 82 - DNA, 19 bp, Artificial Sequence
  Synthetic oligonucleotide
tagcaaacagtgtaaatgc

SEQ ID NO: 83 - DNA, 19 bp, Artificial Sequence
  Synthetic oligonucleotide
gctgctgaatcaaagataa

SEQ ID NO: 84 - DNA, 19 bp, Artificial Sequence
  Synthetic oligonucleotide
ttatctttgattcagcagc

SEQ ID NO: 85 - DNA, 19 bp, Artificial Sequence
  Synthetic oligonucleotide
gccagtgactttgagatta

SEQ ID NO: 86 - DNA, 19 bp, Artificial Sequence
  Synthetic oligonucleotide
taatctcaaagtcactggc

SEQ ID NO: 87 - DNA, 19 bp, Artificial Sequence
  Synthetic oligonucleotide
gctggtccttattgatgaa

SEQ ID NO: 88 - DNA, 19 bp, Artificial Sequence
  Synthetic oligonucleotide
ttcatcaataaggaccagc

SEQ ID NO: 89 - DNA, 19 bp, Artificial Sequence
  Synthetic oligonucleotide
gccctctttgtttccttat

SEQ ID NO: 90 - DNA, 19 bp, Artificial Sequence
  Synthetic oligonucleotide
ataaggaaacaaagagggc

SEQ ID NO: 91 - DNA, 19 bp, Artificial Sequence
  Synthetic oligonucleotide
cagatgaagccttaaataa

SEQ ID NO: 92 - DNA, 19 bp, Artificial Sequence
  Synthetic oligonucleotide
ttatttaaggcttcatctg

SEQ ID NO: 93 - DNA, 19 bp, Artificial Sequence
  Synthetic oligonucleotide
gctctgatgtggaatttaa

SEQ ID NO: 94 - DNA, 19 bp, Artificial Sequence
  Synthetic oligonucleotide
ttaaattccacatcagagc

SEQ ID NO: 95 - DNA, 19 bp, Artificial Sequence
  Synthetic oligonucleotide
ctattacgttcctctataa

SEQ ID NO: 96 - DNA, 19 bp, Artificial Sequence
  Synthetic oligonucleotide
ttatagaggaacgtaatag

SEQ ID NO: 97 - DNA, 19 bp, Artificial Sequence
  Synthetic oligonucleotide
attgatccgattcgattgc

SEQ ID NO: 98 - DNA, 19 bp, Artificial Sequence
  Synthetic oligonucleotide
gcaatcgaatcggatcaat

SEQ ID NO: 99 - DNA, 53 bp, Artificial Sequence
  Synthetic oligonucleotide
ttgtaatttcctgtgcaattggatccaattgcacaggaaattacaactttttc

SEQ ID NO: 100 - DNA, 57 bp, Artificial Sequence
  Synthetic oligonucleotide
tcgagaaaaagttgtaatttcctgtgcaattggatccaattgcacaggaaattacaa

SEQ ID NO: 101 - DNA, 90 bp, Artificial Sequence
  Synthetic oligonucleotide
gttgacagtgagcgcggagagacttggataatgcaatagtgaagccacagatgtattgcattatccaagtctctccttgcctactgcctc

SEQ ID NO: 102 - DNA, 90 bp, Artificial Sequence
  Synthetic oligonucleotide
gttgacagtgagcgcgcgcattatctcagctacttatagtgaagccacagatgtataagtagctgagataatgcgcttgcctactgcctc

SEQ ID NO: 103 - DNA, 90 bp, Artificial Sequence
  Synthetic oligonucleotide
gttgacagtgagcgcccgtagtcactgtatggaatatagtgaagccacagatgtatattccatacagtgactacggttgcctactgcctc

SEQ ID NO: 104 - DNA, 90 bp, Artificial Sequence
  Synthetic oligonucleotide
gttgacagtgagcgtaagcagcgatgtgatttgcattagtgaagccacagatgtaatgcaaatcacatcgctgcttatgcctactgcctc

SEQ ID NO: 105 - DNA, 90 bp, Artificial Sequence
  Synthetic oligonucleotide
gttgacagtgagcgtgatctagttacagctgaagcatagtgaagccacagatgtatgcttcagctgtaactagatcatgcctactgcctc

SEQ ID NO: 106 - DNA, 90 bp, Artificial Sequence
  Synthetic oligonucleotide
gttgacagtgagcgtggacatttactggctcatgattagtgaagccacagatgtaatcatgagccagtaaatgtccatgcctactgcctc

SEQ ID NO: 107 - DNA, 90 bp, Artificial Sequence
  Synthetic oligonucleotide
gttgacagtgagcgcggactttatcagaaactcaaatagtgaagccacagatgtatttgagtttctgataaagtccttgcctactgcctc

SEQ ID NO: 108 - DNA, 90 bp, Artificial Sequence
  Synthetic oligonucleotide
gttgacagtgagcgcgccacgtttcaacagatatattagtgaagccacagatgtaatatatctgttgaaacgtggcttgcctactgcctc

SEQ ID NO: 109 - DNA, 90 bp, Artificial Sequence
  Synthetic oligonucleotide
gttgacagtgagcgcggagtctgcgtgcgaggattatagtgaagccacagatgtataatcctcgcacgcagactccttgcctactgcctc

SEQ ID NO: 110 - DNA, 90 bp, Artificial Sequence
  Synthetic oligonucleotide
gttgacagtgagcgaaaccttcctatgatattgatatagtgaagccacagatgtatatcaatatcataggaaggttgtgcctactgcctc

SEQ ID NO: 111 - DNA, 90 bp, Artificial Sequence
  Synthetic oligonucleotide
gttgacagtgagcgcggtgtccattacttcaatatttagtgaagccacagatgtaaatattgaagtaatggacaccatgcctactgcctc

SEQ ID NO: 112 - DNA, 90 bp, Artificial Sequence
  Synthetic oligonucleotide
gttgacagtgagcgaaggttatctgtgctacaattgtagtgaagccacagatgtacaattgtagcacagataacctgtgcctactgcctc

SEQ ID NO: 113 - DNA, 90 bp, Artificial Sequence
  Synthetic oligonucleotide
gttgacagtgagcgcgcagatcatgtgtgagctaaatagtgaagccacagatgtatttagctcacacatgatctgcatgcctactgcctc